(19) United States
(12) Patent Application Publication
(10) Pub. No.: US 2007/0016982 A1
(43) Pub. Date: Jan. 18, 2007

(54) Transgenic plants with increased titerpene saponin levels

(76) Inventors: Carl A. Maxwell, Elkton, MD (US); Brian McGonigle, Wilmington, DE (US); Aideen Oonagh Hession, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/484,996

(22) Filed: Jul. 12, 2006

Related U.S. Application Data
(60) Provisional application No. 60/699,135, filed on Jul. 13, 2005.

(51) Int. Cl. A01H 1/00; C12N 15/82; C12N 5/04; A61K 31/704

(52) U.S. Cl. 800/284; 514/26; 514/33; 435/419; 435/468

(57) ABSTRACT

The invention relates to plants with increased levels of triterpene saponins. The plant may have an increased activity of at least one oxidosqualene cyclase that catalyzes the cyclization of 2,3-oxidosqualene to form a cyclyzed triterpene. The plant may be transformed with at least one recombinant DNA molecule comprising a promoter operably linked to at least one polynucleotide encoding an oxidosqualene cyclase, said recombinant DNA molecule sufficient to increase production of a triterpene saponin; or any progeny of said plant, wherein said progeny comprise said recombinant DNA molecule.

This application claims the benefit of U.S. Provisional Application No. 60/699,135 filed Jul. 13, 2005, the disclosure of which is hereby incorporated by reference in its entirety.

### FIELD OF THE INVENTION

This invention is in the field of plant molecular biology. More specifically, this invention pertains to plants with increased levels of triterpene saponins. The plant may have an increased activity of at least one oxidosqualene cyclase that catalyzes the cyclization of 2,3-oxidosqualene to form a cyclyzed triterpene. The plant may be transformed with at least one recombinant DNA molecule comprising a promoter operably linked to at least one polynucleotide encoding an oxidosqualene cyclase, said recombinant DNA molecule sufficient to increase production of triterpene saponin; or any progeny of said plant, wherein said progeny comprise said recombinant DNA molecule. Protein products, as well as food and feed products obtained from plants and/or seeds having an increased triterpene saponin level are also part of the invention.

### BACKGROUND OF THE INVENTION

The terpenes, which are composed of one or more five-carbon isoprene units, constitute the largest family of natural products with over 22,000 individual compounds of this class having been described. The terpenes (hemiterpenes, monoterpenes, sesquiterpenes, diterpenes, triterpenes, tetraterpenes, polyterpenes, and the like) play diverse functional roles in plants as hormones, photosynthetic pigments, electron carriers, mediators of polysaccharide assembly, and structural components of membranes. Plant terpenes are found in resins, latex, waxes, and oils.

Triterpenes, as well as sterols, are biosynthesized via the isoprenoid pathway. In this pathway, two molecules of farnesyl pyrophosphate are joined head-to-head to form squalene, a triterpene. Squalene is then converted to 2,3-oxidosqualene. Various oxidosqualene cyclases catalyze the cyclization of 2,3-oxidosqualene to form various polycyclic skeletons, including one or more of cycloartenol, lanosterol, lupeol, isomultiflorenol, β-amyrin, α-amyrin, and thalianol. This cyclization event catalyzed by oxidosqualene cyclases forms a branch point between the sterol and triterpene saponin biosynthetic pathways. The various oxidosqualene cyclases are evolutionarily related (Kushiro, T., et al. (1998) Eur. J. Biochem. 256:238-244) and produce a wide variety of three-, four-, and five-ring structures that can be further modified.

For sterol synthesis, the cyclization of 2,3-oxidosqualene is catalyzed by the 2,3-oxidosqualene cyclases, cycloartenol synthase and lanosterol synthase. Cycloartenol (in photosynthetic organisms) and lanosterol (in non-photosynthetic organisms) are 30 carbon, 4-ring structures that can be further modified to form sterols. In photosynthetic organisms, sterols have a wide range of functions including regulation of membrane fluidity and as precursors for the brassinosteroids. In some plants, sterols can also be glycosylated to form steroidal saponins.

For triterpene saponin synthesis, the cyclization of 2,3-oxidosqualene is catalyzed by 2,3-oxidosqualene cyclases, such as lupeol synthase, β-amyrin synthase, α-amyrin synthase, isomultiflorenol synthase, thalianol synthase and dammarenediol synthase. Lupeol, β-amyrin, α-amyrin, isomultiflorenol and thalianol, can be further modified (e.g., oxidation, substitution, and glycosylation) to form triterpene saponins. For example, the basic β-amyrin ring structure may be modified by glycosylation (sometimes preceded by hydroxylation) to form triterpene saponins. The function of triterpene saponins is unclear although it is thought that they play a defense role against pathogens in plant tissues. Triterpene saponins may also confer resistance to insects.

Soybean saponins are triterpene glycosides and comprise triterpenes linked to one or more hydrophilic mono- or oligosaccharide moieties. Saponins, in soybean, are classified into two major groups, saponin A and B. Group A saponins are implicated as contributing to an undesirable bitter and astringent taste. Group B saponins are implicated as having several health benefits. They appear to possess inhibitory activity against infection by human immunodeficiency virus (HIV) (Nakashima, H., et al. (1989) AIDS 3:655-658) and the activation of Epstein-Barr virus early antigen (Konoshima et al. (1991) J. Nat. Prod. 54:830-836). Group B saponins have also been suggested to possess hypocholesterolemic, immunostimulatory, anticarcinogenic, antioxidative, antitumor, antivirus, antihepatitic, antidiabetic, and hepatoporotective properties (Fournier, et al., (1998) Cancer Epidemiol. Biomarkers Prev. 7:1055-1065.)

There is an accumulating amount of data suggesting that saponins in the diet may be beneficial (see for example Shi, J. A. et al. (2004) J. Med. Food 7:67-78 and Vis, E. H. et al. (2005) Nutr. Cancer 51:37-44). For instance, Vis et al. state “data suggest a protective effect from soy saponins by reducing lytic activity of cholic acid.” Similarly, dietary saponins of soybean have been shown to be beneficial in preventing hypercholesterolemia and aortic atherosclerosis in rats (Oakenfull, et al. (1984) Nutr. Rep. Int. 29:1039-1046). Since saponins are carried over from the bean into soy isolate with only minimal loss, increased levels of saponins in beans should lead to increased amounts of saponins in isolate (Berhow, M. A. et al. (2002) Phytochem. Anal. 13:343-348; Hu J., et al. (2002) J. Agric. Food Chem. 50:2587-2594). Increasing levels of saponins in beans, thus, would be an effective way of increasing saponin amounts in the human diet. Total triterpene saponin content varies somewhat by soybean cultivar but is in the range of 0.25% of the seed dry weight (Shiraiwa, M., et al. (1991) Agric. Biol. Chem. 55:323-331).

Other benefits of increased saponins in plants could improve disease resistance, in particular fungal resistance, and herbivore resistance. In addition, the increase in saponins could provide an source for compounds used in drug development.

PCT Publication WO 01/66,773, published Sep. 13, 2001 discloses polynucleotides encoding oxidosqualene cyclases from soybean and wheat. This publication discloses production of detectable levels of β-amyrin when expressing the cDNA insert of clone src3c.pk024.m11 in yeast.

PCT Publication WO 03/095,615, published Nov. 11, 2003 discloses lowering triterpene saponin levels in plants and seeds by transforming a plant cell with at least a portion of at least one oxidosqualene cyclase gene, said portion being sufficient to suppress production of triterpene saponins.

### SUMMARY OF THE INVENTION

The present invention involves a plant with increased levels of triterpene saponins comprising an increased activity of at least one oxidosqualene cyclase that catalyzes the cyclization of 2,3-oxidosqualene to form a triterpene, wherein the increased activity of the oxidosqualene cyclase is sufficient to increase triterpene saponin levels in the plant. In a preferred embodiment, the oxidosqualene cyclase catalyzes the cyclization of 2,3-oxidosqualene to form at least one triterpene selected from the group consisting of β-amyrin, lupeol, α-amyrin, isomultiflorenol, thalianol, and any combination thereof. In a more preferred embodiment, the oxidosqualene cyclase is selected from the group consisting of lupeol synthase, β-amyrin synthase, α-amyrin synthase, isomultiflorenol synthase, thalianol synthase and dammarenediol synthase. In an even more preferred embodiment, the oxidosqualene cyclase is a β-amyrin synthase. In an even more preferred embodiment, the β-amyrin synthase has the amino acid sequence of SEQ ID NO:11.

In another embodiment, the plant is transformed by at least one recombinant DNA molecule comprising a promoter operably linked to at least one polynucleotide encoding an oxidosqualene cyclase, said recombinant DNA molecule sufficient to increase triterpene saponin levels; or any progeny of said plant, wherein said progeny comprise said recombinant DNA molecule. In a preferred embodiment, the invention relates to a plant transformed with at least one recombinant DNA molecule comprising an oxidosqualene cyclase that catalyzes cyclization of 2,3-oxidosqualene to form at least one triterpene selected from the group consisting of β-amyrin, lupeol, α-amyrin, isomultiflorenol, thalianol, and any combination thereof. In a more preferred embodiment, the oxidosqualene cyclase is selected from the group consisting of lupeol synthase, β-amyrin synthase, α-amyrin synthase, isomultiflorenol synthase, thalianol synthase and dammarenediol synthase. In an even more preferred embodiment, the oxidosqualene cyclase is a β-amyrin synthase. In an even more preferred embodiment, the β-amyrin synthase has the amino acid sequence of SEQ ID NO:11.

In another embodiment, the invention concerns a plant transformed with at least one recombinant DNA molecule comprising a promoter selected from the group consisting of a seed-specific promoter, a root-specific promoter, a vacuole-specific promoter, a leaf-specific promoter, a pod-specific promoter, and an embryo-specific promoter. In a preferred embodiment, the promoter is a seed-specific promoter.

In another embodiment, the invention relates to a transformed plant or plant part having increased triterpene saponin levels, wherein said plants are selected from the group consisting of soybean, alfalfa, peanut, pea, lentil, chick pea, kidney bean, pigeon pea, oat, and wheat. In a preferred embodiment, the plant is soybean. Seeds from said plant are also part of the invention. Preferentially said seeds are soybean seeds. Also part of the invention are feed and food, including beverages, protein products, and industrial products having increased triterpene saponin levels and prepared from said seeds.

The invention also concerns a method for increasing the triterpene saponin level in a plant comprising: (a) transforming a plant cell with a nucleic acid fragment that increases the activity of at least one oxidosqualene cyclase that catalyzes the cyclization of 2,3-oxidosqualene to form a triterpene to produce a transformed plant cell; (b) growing said transformed plant cell from step (a) under conditions that promote the regeneration of a transgenic plant; and (c) evaluating the transgenic plant of step (b) for an increased level of triterpene saponin when compared to the amount of triterpene saponin in a plant of the same species that is not transformed with said nucleic acid fragment. In a preferred embodiment the plant is a soybean plant.

The invention also concerns a method for increasing the triterpene saponin level in a plant comprising: (a) creating a recombinant DNA molecule comprising a promoter operably linked to at least one polynucleotide encoding an oxidosqualene cyclase that catalyzes the cyclization of 2,3-oxidosqualene to form a triterpene; (b) transforming a plant cell with said recombinant DNA molecule to produce a transformed plant cell; (c) growing said transformed plant cell from step (b) under conditions that promote the regeneration of a transgenic plant, and (d) evaluating the transgenic plant of step (c) for an increased level of triterpene saponin when compared to the amount of triterpene saponin in a plant of the same species that is not transformed with said recombinant DNA molecule.

In another embodiment, the invention relates to a method for increasing the triterpene saponin level in a plant selected from the group consisting of soybean, alfalfa, peanut, pea, lentil, chickpea, pigeon pea, oat, and wheat. In a preferred embodiment, the plant is soybean.

In another embodiment, the invention concerns a transgenic plant or plant part prepared by the method of the invention. Also included are seed derived from such plant. It is preferred that the plant be a soybean plant. Also part of the invention are feed and food, including beverages, and industrial products having increased triterpene saponin levels and prepared from seeds obtained from transgenic plants or plant parts prepared by the method of the invention.

In another embodiment, the invention relates to a plant transformed with a recombinant DNA molecule comprising a seed-specific promoter operably linked to a DNA fragment encoding β-amyrin synthase, said recombinant DNA molecule sufficient to increase production of a triterpene saponin; or any progeny of said plant wherein said progeny comprise said recombinant DNA molecule. In a preferred embodiment, the β-amyrin synthase has the amino acid sequence of SEQ ID NO:11.

In another embodiment, the invention concerns a method for increasing the triterpene saponin level in a transgenic soybean plant comprising: (a) creating a recombinant DNA molecule comprising a seed specific promoter operably linked to a DNA fragment encoding a β-amyrin synthase having the amino acid sequence of SEQ ID NO:11; (b) transforming a soybean plant cell with said recombinant DNA molecule to produce a transformed plant cell; (c) growing said transformed plant cell from step (b) under conditions that promote regeneration of a transgenic plant, and (d) evaluating the transgenic plant of step (c) for an amount of triterpene saponin that is increased when compared to the amount of triterpene saponin in a soybean plant that is not transformed with said recombinant DNA molecule.

In still another embodiment, the invention concerns a method of producing a high-triterpene saponin product which comprises: (a) cracking the seeds obtained from a plant with an increased activity of at least one oxidosqualene cyclase to remove the meats from the hulls; and (b) flaking the meats obtained in step (a) to obtain the desired flake thickness. Also part of the invention are feed and food, including beverages, protein products, and industrial products having increased triterpene saponin levels prepared by the method of the invention. In a preferred embodiment, the plant is a soybean plant.

### BRIEF DESCRIPTION OF THE FIGURE AND SEQUENCE LISTINGS

The invention can be more fully understood from the following detailed description and the accompanying drawing and Sequence Listing which form a part of this application.

FIG. 1: FIG. 1 shows the saponin biosynthetic pathway.

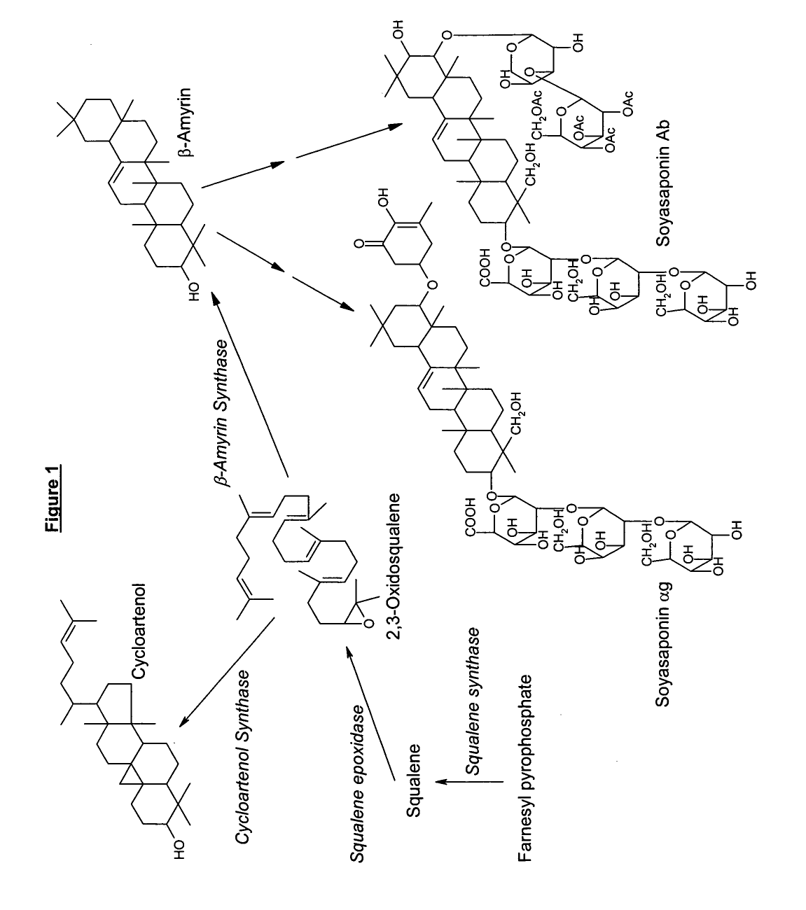

The sequence descriptions and Sequence Listing attached hereto comply with the rules governing nucleotide and/or amino acid sequence disclosures in patent applications as set forth in 37 C.F.R. §1.821-1.825.

SEQ ID NO:1 is the nucleotide sequence of the 6383 bp fragment that is flanked by Kpn I restriction endonuclease digestion sites and contains a plant selectable marker gene cassette and a cassette comprising a promoter and terminator separated by a unique Not I restriction endonuclease site.

SEQ ID NO:2 is the deduced amino acid sequence of the mutant soybean ALS having alanine instead of proline at position 183 and leucine instead of tryptophan at position 560.

SEQ ID NO:3 is the nucleotide sequence of plasmid pKS210.

SEQ ID NO:4 is the nucleotide sequence of oligonucleotide primer BM5.

SEQ ID NO:5 is the nucleotide sequence of oligonucleotide primer BM6.

SEQ ID NO:6 is the nucleotide sequence of plasmid pDN10.

SEQ ID NO:7 is the nucleotide sequence of the cDNA insert in clone src3c.pk024.m11 encoding α-amyrin synthase.

SEQ ID NO:8 is the nucleotide sequence of oligonucleotide primer BM7.

SEQ ID NO:9 is the nucleotide sequence of oligonucleotide primer BM8.

SEQ ID NO:10 is the nucleotide sequence of the amplification product encoding a β-amyrin synthase obtained by amplifying the cDNA insert in clone src3c.pk024.m11 with oligonucleotide primers BM7 and BM8.

SEQ ID NO:11 is the amino acid sequence encoded by SEQ ID NO:10.

SEQ ID NO:12 is the nucleotide sequence of plasmid PHP20767.

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984) which are herein incorporated by reference. The symbols and format used for nucleotide and amino acid sequence data comply with the rules set forth in 37 C.F.R. §1.822.

### Definitions

In the context of this disclosure, a number of terms shall be utilized.

The terms “recombinant DNA molecule,” “recombinant DNA fragment,” “recombinant DNA expression cassette,” “recombinant construct,” “expression construct,” “chimeric construct,” and “recombinant DNA construct,” are used interchangeably herein and are nucleic acid fragments. A recombinant construct comprises an artificial combination of nucleic acid fragments, including, and not limited to, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such construct may be used by itself, may be used with at least one other recombinant DNA construct, or may be used in conjunction with a vector.

A “vector” is a polynucleotide fragment to which at least one fragment of DNA from a different organism may be integrated and, which, when introduced into a host cell is capable of either self-replicating or integrating itself in the host chromosome. The choice of vector is dependent upon the method used to transform host cells as is well known to those skilled in the art. Screening to obtain lines displaying the desired phenotype may be accomplished by Southern analysis of DNA, Northern analysis of mRNA expression, RT-PCR, immunoblotting analysis of protein expression, or phenotypic analysis, among others.

As used herein, the term “cassette” refers to a recombinant DNA construct comprising one or more polynucleotides of interest, which is flanked by restriction endonuclease sites so that it may be removed from other fragments in the same construct. The restriction endonuclease sites flanking the recombinant DNA construct may be the same at, both, the 5′ and 3′ ends of the polynucleotide or may be different.

The terms “polynucleotide” and “nucleic acid fragment” are used interchangeably herein. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof.

“Codon degeneracy” refers to divergence in the genetic code permitting variation of the nucleotide sequence without affecting the amino acid sequence of an encoded polypeptide. Accordingly, the instant invention relates to any nucleic acid fragment comprising a nucleotide sequence that encodes all or a substantial portion of the amino acid sequences set forth herein. The skilled artisan is well aware of the “codon-bias” exhibited by a specific host cell in usage of nucleotide codons to specify a given amino acid. Therefore, when synthesizing a nucleic acid fragment for improved expression in a host cell, it is desirable to design the nucleic acid fragment such that its frequency of codon usage approaches the frequency of preferred codon usage of the host cell.

“Synthetic nucleic acid fragments” can be assembled from oligonucleotide building blocks that are chemically synthesized using procedures known to those skilled in the art. These building blocks are ligated and annealed to form larger nucleic acid fragments which may then be enzymatically assembled to construct the entire desired nucleic acid fragment. “Chemically synthesized”, as related to a nucleic acid fragment, means that the component nucleotides were assembled in vitro. Manual chemical synthesis of nucleic acid fragments may be accomplished using well established procedures, or automated chemical synthesis can be performed using one of a number of commercially available machines. Accordingly, the nucleic acid fragments can be tailored for optimal gene expression based on optimization of the nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

“Gene” refers to a nucleic acid fragment that expresses a specific protein or RNA, including regulatory sequences preceding (5′ non-coding sequences) and following (3′ non-coding sequences) the coding sequence. “Native gene” refers to a gene as found in nature with its own regulatory sequences. “Chimeric gene” refers any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign-gene” refers to a gene not normally found in the host organism, but that is introduced into the host organism by gene transfer. Foreign genes can comprise native genes inserted into a non-native organism, recombinant DNA constructs, or chimeric genes. A “transgene” is an isolated nucleic acid fragment or recombinant DNA construct that has been introduced into the genome by a transformation procedure.

“Coding sequence” refers to a nucleotide sequence that encodes a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

“Promoter” refers to a region of DNA capable of controlling the expression of a coding sequence or functional RNA. The promoter may consist of proximal and more distal upstream elements. These upstream elements include, but are not limited to, enhancers, repressor binding motifs, tissue-specific motifs, developmental responsive motifs, and hormone responsive motifs. An “enhancer” is a region of DNA capable of stimulating promoter activity. These upstream elements may be innate regions of the promoter or a heterologous element inserted to enhance the level or tissue-specificity of a promoter.

A number of promoters can be used in the practice of the present invention. The promoters can be selected based on the desired outcome. Nucleic acid fragments used to accomplish the invention can be combined in any host organism with a promoter or element that has constitutive, tissue-specific, inducible, or other gene regulatory activities.

In some embodiments, promoters or enhancers can be used or modified to accomplish the present invention. For example, endogenous promoters can be altered in vivo by mutation, deletion, and/or substitution (see for example U.S. Pat. No. 5,565,350). Gene expression can be modulated under conditions suitable for host cell growth so as to alter the total concentration and/or alter the composition of the polypeptides of the present invention in host cell.

“Tissue-specific” promoters preferentially direct RNA production in particular types of cells or tissues. Promoters that cause a gene to be expressed in most cell types at most times are commonly referred to as “constitutive promoters.” New promoters of various types useful in plant cells are constantly being discovered; the compilation by Okamuro, J. K. and Goldberg, R. B. (1989, Biochemistry of Plants 15:1-82) provides numerous examples. It is further recognized that since in most cases the exact boundaries of regulatory sequences have not been completely defined, DNA fragments of some variation may have identical promoter activity.

Commonly used promoters include, but are not limited to, the nopaline synthase (NOS) promoter (Ebert et al. (1987) Proc. Natl. Acad. Sci. U.S.A. 84:5745-5749), the octapine synthase (OCS) promoter, caulimovirus promoters such as the cauliflower mosaic virus (CaMV) 19S promoter (Lawton et al. (1987) Plant Mol. Biol. 9:315-324), the CaMV 35S promoter (Odell et al. (1985) Nature 313:810-812), and the figwort mosaic virus 35S promoter; the light inducible promoter from the small subunit of rubisco, the Adh promoter (Walker et al. (1987) Proc. Natl. Acad. Sci. U.S.A. 84:6624-66280), the sucrose synthase promoter (Yang et al. (1990) Proc. Natl. Acad. Sci. U.S.A. 87:4144-4148), the R gene complex promoter (Chandler et al. (1989) Plant Cell 1:1175-1183), and the chlorophyll a/b binding protein gene promoter. Other commonly used promoters are, the promoters for the potato tuber ADPGPP genes, the granule bound starch synthase promoter, the glutelin gene promoter, the maize waxy promoter, the Brittle gene promoter, the Shrunken 2 promoter, the acid chitinase gene promoter, and the zein gene promoters (15 kD, 16 kD, 19 kD, 22 kD, and 27 kD; Perdersen et al. (1982) Cell 29:1015-1026). A plethora of promoters is described in PCT Publication No. WO 00/18963, published on Apr. 6, 2000.

The “translation leader sequence” or “leader” refers to a polynucleotide sequence located upstream or 5′ of the coding sequence. The translation leader sequence is present in the fully processed mRNA upstream of the translation start site. The translation leader sequence may affect processing of the primary transcript to mRNA, mRNA stability or translation efficiency. Examples of translation leader sequences have been described (Turner, R. and Foster, G. D. (1995) Mol. Biotechnol. 3:225-236).

The “3′ non-coding region” and “terminator region” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al. (1989, Plant Cell 1:671-680).

The term “operably linked” and “under the control of” refer to the association of nucleic acid fragments on a single polynucleotide so that the function of one is affected by the function of the other. For example, a promoter is operably linked with a coding sequence when it is capable of affecting the expression of that coding sequence (i.e., that the coding sequence is under the transcriptional control of the promoter). Similarly, a polynucleotide may be under the control of a promoter that is capable of affecting the expression of the polynucleotide. Coding sequences can be operably linked to regulatory sequences in sense or antisense orientation.

“RNA transcript” refers to the product resulting from RNA polymerase-catalyzed transcription of a DNA sequence. When the RNA transcript is a perfect complementary copy of the DNA sequence, it is referred to as the primary transcript. An RNA sequence derived from posttranscriptional processing of the primary transcript and is referred to as the mature RNA. “Messenger RNA (mRNA)” refers to the RNA that is without introns and that can be translated into protein by the cell. “cDNA” refers to a DNA that is complementary to and derived from an mRNA. The cDNA can be single-stranded or converted into the double stranded form using, for example, the Klenow fragment of DNA polymerase I. “Sense” RNA refers to an RNA transcript that includes the mRNA and can be translated into a polypeptide by the cell. “Antisense RNA” refers to an RNA transcript that is complementary to all or part of a target primary transcript or mRNA and that blocks the expression of a target gene (see U.S. Pat. No. 5,107,065). The complement of an antisense RNA may be with any part of the specific gene transcript, i.e., at the 5′ non-coding sequence, 3′ non-coding sequence, introns, or the coding sequence. “Functional RNA” refers to sense RNA, antisense RNA, ribozyme RNA, or other RNA that may not be translated but yet has an effect on cellular processes.

The terms “PCR,” “polymerase chain reaction,” and “PCR amplification” are used interchangeably herein and refer to a technique for the synthesis of large quantities of specific DNA fragments. It is well known by those skilled in the art as a technique used for the amplification of specific DNA segments (U.S. Pat. Nos. 4,683,195 and 4,800,159).

The term “transformation” refers to the transfer of a nucleic acid fragment into the genome of a host organism. Host organisms containing a transferred nucleic acid fragment are referred to as “transgenic” or “transformed” organisms. “Transgenic” or “transformed” organisms include the originally transformed organisms as well as any progeny thereof that contains the transferred nucleic acid fragment. “Host cell” refers the cell into which a nucleic acid fragment is transferred and may include a yeast cell, a bacterial cell, an insect cell, or a plant cell. Examples of methods of plant transformation include, among others, Agrobacterium-mediated transformation (De Blaere et al. (1987) Meth. Enzymol. 143:277) and particle-accelerated transformation (Klein et al. (1987) Nature (London) 327:70-73; U.S. Pat. No. 4,945,050). Particle accelerated transformation is also referred to as “gene gun” transformation.

There are a variety of methods for the regeneration of plants from plant tissue. The particular method of regeneration will depend on the starting plant tissue and the particular plant species to be regenerated.

The regeneration, development and cultivation of plants from single transformed plant cells or from various transformed explants is well known in the art (Weissbach and Weissbach, In.: Methods for Plant Molecular Biology, (Eds.), Academic Press, Inc., San Diego, Calif. (1988)). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The term “T0 plant” refers to a primary transformant regenerated from the initially transformed host cell. The term “T1 seed” refers to the seed produced by a “T0 plant.”

The term “progeny” refers to the plants and seed obtained after selfing or crossing a plant of interest. The first generation progeny from T0 plants are referred to as “T1 plants”, the next generation is referred to as “T2 plants” and so on.

Preferably, the regenerated plants are self-pollinated to provide homozygous transgenic plants. Pollen obtained from the regenerated plants may also be crossed to plants of agronomically important lines. Conversely, pollen from plants of these agronomically important lines is used to pollinate regenerated plants. A transgenic plant of the present invention, comprising an increased activity of at least one oxidosqualene cyclase sufficient to increase the levels of triterpene saponins in said transgenic plant, is cultivated using methods well known to one skilled in the art.

The term “event” refers to a unique incidence of transformation and multiple, identical plants can be regenerated from a single event.

The term “expression,” as used herein refers to the transcription and stable accumulation of mRNA or RNA derived from a polynucleotide of the invention. Expression may also refer to translation of mRNA into a polypeptide.

The terms “altered levels” and “altered expression” refer to the production of gene product(s) in transgenic organisms in amounts or proportions that differ from that of non-transformed organisms or organisms transformed with nucleic acid fragments other than those in the current invention.

The term “overexpression” refers to the production of a gene product in a transgenic organism that exceeds levels of production in an organism not transformed with the recombinant DNA or nucleic acid fragment of the invention.

Standard recombinant DNA and molecular cloning techniques used herein are well known in the art and are described more fully in Sambrook et al. Molecular Cloning: A Laboratory Manual; Cold Spring Harbor Laboratory Press: Cold Spring Harbor, 1989.

### DETAILED DESCRIPTION OF THE INVENTION

The present invention relates to a plant with an increased level of triterpene saponins. The plant may have an increased activity of at least one oxidosqualene cyclase, wherein the increased activity of the oxidosqualene cyclase is sufficient to increase triterpene saponin levels in said plant. The oxidosqualene cyclase catalyzes the cyclization of 2,3-oxidosqualene to form a triterpene.

In the present invention, a plant may be transformed with at least one recombinant DNA molecule comprising a promoter operably linked to at least one polynucleotide encoding an oxidosqualene cyclase, said recombinant DNA molecule sufficient to increase triterpene saponin levels; or any progeny of said plant, wherein said progeny comprise said recombinant DNA molecule. The recombinant DNA molecule of the instant invention is used to create transgenic plants in which the triterpene saponin content is increased with respect to a plant not containing said recombinant DNA molecule. The corresponding changes in the resulting plant and seed are useful to improve their nutritional and health value.

In the present invention, a plant may be transformed with a nucleic acid fragment capable of increasing the activity of at least one oxidosqualene cyclase said nucleic acid fragment sufficient to increase triterpene saponin levels; or any progeny of said plant, wherein said progeny comprise said nucleic acid fragment. The nucleic acid fragment may increase the activity of the oxidosqualene cyclase by enhancing the levels of at least one endogenous oxidosqualene cyclase gene, for example, by gene activation. Accordingly, the nucleic acid fragment may comprise an enhancer or relevant transcription factor. Also in the present invention, the activity of at least one oxidosqualene cyclase may be increased by stabilizing or increasing translation of the mRNA encoding the oxidosqualene cyclase.

Oxidosqualene cyclases are a large family of enzymes that catalyze cyclization of 2,3-oxidosqualene to form various polycyclic skeletons including one or more of lanosterol, cycloartenol, lupeol, isomultiflorenol, β-amyrin, α-amyrin, and thalianol, or combination thereof. Oxidosqualene cyclases include, and are not limited to, cycloartenol synthase, lanosterol synthase, β-amyrin synthase, lupeol synthase, mixed amyrin synthase, isomultiflorenol synthase, thalianol synthase, dammarenediol synthase. Additionally, there are oxidosqualene cyclases that synthesize cucurbita-5,24-dienol, parkeol, protosta13(17),24-dien-3-ol and protosta-17(20),24-dien-3-ol. There are 83 triterpene alcohols that are structurally consistent with being enzymatic cyclization products of oxidosqualene. It is not known if there would be a separate oxidosqualene cyclase for each compound (Matsuda, On the diversity of Oxidosqualene cyclases. Biochemical Principles and Mechanisms of Biosynthesis and biodegradation of Polymers. A. Steinbuchel, Ed., Wiley-VCH, 1998; Xu et al.(2004). Phytochemistry. 65:261-291).

Oxidosqualene cyclases useful in the present invention are a subset of the family that preferentially catalyze the cyclization of 2,3-oxidosqualene to form triterpenes which are intermediates along the biosynthetic pathway to form triterpene saponins. Examples of such oxidosqualene cyclases include, and are not limited to, β-amyrin synthase, lupeol synthase, mixed amyrin synthase, isomultiflorenol synthase, thalianol synthase, dammarenediol synthase and the like. Also useful in the present invention are fragments and hybrids thereof. β-amyrin synthase, which catalyzes the cyclization of 2,3-oxidosqualene to β-amyrin, is a preferred example of an oxidosqualene cyclase useful in the present invention. Other examples of oxidosqualene cyclases are disclosed in U.S. Patent Publication 20030208791, which is incorporated by reference, and by Iturbe-Ormaetxe et al., (2003) Plant Mol. Biol. 51:731-743.

In a preferred embodiment, the oxidosqualene cyclase is a β-amyrin synthase. β-amyrin synthases have been functionally characterized from Panax ginseng (Kushiro, T., et al. (1998) Eur. J. Biochem. 256:238-244); pea (Morita, M., et al. (2000) Eur. J. Biochem. 267: 3453-3460), oat (Haralampidis, K., et al. (2001) Proc. Natl. Acad. Sci. U.S.A. 98:13431-13436), soybean (PCT Publication WO 01/66,773, published Sep. 13, 2001), Glycyrrhiza glabra (Hayashi, H. et al. (2001) Biol. Pharm. Bull. 24:912-916), Betula platyphylla (Zhang, H. et al. (2003) Biol. Pharm. Bull. 26:642-650), and Medicago truncatula (Iturbe-Ormaetxe, I., et al. (2003) Plant Mol. Biol. 51:731-743). In an even more preferred embodiment, the β-amyrin synthase has the amino acid sequence set forth in SEQ ID NO:11. The amino acid sequence set forth in SEQ ID NO:11 was obtained by translating nucleotides 9 through 2294 of SEQ ID NO:10. The nucleotide sequence set forth in SEQ ID NO:10 is the nucleotide sequence of the amplification product encoding a β-amyrin synthase obtained by amplifying the cDNA insert in clone src3c.pk024.m11 with oligonucleotide primers BM7 (SEQ ID NO:8) and BM8 (SEQ ID NO:9).

A polynucleotide encoding an oxidosqualene cyclase useful in the invention may have substantial similarity to a polynucleotide encoding the amino acid sequence set forth in SEQ ID NO:11. The oxidosqualene cyclase encoded by the polynucleotide useful in the invention catalyzes the cyclization of 2,3-oxidosqualene to form various polycyclic skeletons resulting in the formation of triterpene saponins. It may encode an oxidosqualene cyclase at least 80% identical to the amino acid sequence set forth in SEQ ID NO 11. It is preferred that the identity be at least 90%, it is preferable if the identity is at least 95% or any integer thereof (see for example Kushiro et al. (2000) J. Am. Chem. Soc. 122:6816-6824).

Any promoter can be used in accordance with the method of the invention. Thus, the origin of the promoter chosen to drive expression of the coding sequence is not critical as long as it has sufficient transcriptional activity to accomplish the invention by expressing translatable mRNA for the desired genes in the desired host tissue. The promoter for use in the present invention may be selected from the group consisting of a seed-specific promoter, a root-specific promoter, a vacuole-specific promoter, a leaf-specific promoter, a pod-specific promoter, and an embryo-specific promoter.

Examples of a seed-specific promoter include, but are not limited to, the promoter for β-conglycinin (Chen et al. (1989) Dev. Genet 10: 112-122), the napin promoter, and the phaseolin promoter. Other tissue-specific promoters that may be used to accomplish the invention include, but are not limited to, the chloroplast glutamine synthase (GS2) promoter (Edwards et al. (1990) Proc. Natl. Acad. Sci. U.S.A. 87:3459-3463), the chloroplast fructose-1,6-biophosphatase promoter (Lloyd et al. (1991) Mol. Gen. Genet. 225:209-2216), the nuclear photosynthetic (ST-LS1) promoter (Stockhaus et al. (1989) EMBO J. 8:2445-2451), the serine/threonine kinase (PAL) promoter, the glucoamylase promoter, the promoters for the Cab genes (cab6, cab-1, and cab-1R; Yamamoto et al. (1994) Plant Cell Physiol. 35:773-778; Fejes et al. (1990) Plant Mol. Biol. 15:921-932; Lubberstedt et al. (1994) Plant Physiol. 104:997-1006; Luan et al. (1992) Plant Cell 4:971-981), the pyruvate orthophosphate dikanase promoter (Matsuoka et al. (1993) Proc. Natl. Acad. Sci. U.S.A. 90:9586-9590), the LhcB promoter (Cerdan et al. (1997) Plant Mol. Biol. 33:245-255), the PsbP promoter (Kretsch et al. (1995) Plant Mol. Biol. 28:219-229), the SUC2 sucrose H+ symporter promoter (Truernit et al. (1995) Planta 196:564-570), and the promoters for the thylakoid membrane genes (psaD, psaF, psaE, PC, FNR, atpC, atpD), etc.

More specifically, a preferred embodiment of the invention relates to a plant transformed with a recombinant DNA molecule comprising a seed specific promoter operably linked to a DNA fragment encoding a β-amyrin synthase having the amino acid sequence of SEQ ID NO:11, said recombinant DNA molecule sufficient to increase production of a triterpene saponin; or any progeny of said plant wherein said progeny comprise said recombinant DNA molecule. The transformed plant or any progeny of said transformed plant comprising the recombinant DNA molecule sufficient to increase the triterpene saponins in the plant is then grown under conditions suitable for the expression of the recombinant DNA molecule. Expression of the recombinant DNA molecule increases triterpene saponin content of the transformed plant compared to the triterpene saponin content of a non-transformed plant or a plant transformed with recombinant constructs other than those in the current invention.

“Saponins” refers to the glycoside conjugates of cyclized triterpenes that naturally accumulate in plants. Cyclized triterpenes include, and are not limited to, lanosterol, cycloartenol, β-amyrin, α-amyrin, lupeol, isomultiflorenol, and thalianol. “Triterpene saponins” refers to the glycoside conjugates of cyclized triterpenes excluding those derived from lanosterol or cycloartenol. “Steroidal saponins” refer to the glycoside conjugates derived from lanosterol or cycloartenol. Sapogenols are derived from triterpene saponins via in vitro acid hydrolysis and their measurement provides a relative value for the amount of triterpene saponins present in the tissue from which the saponins are extracted.

The “increased triterpene saponin levels,” for purposes of the present invention refer to triterpene saponin levels higher than those found in non-transformed plants of the same species not having an increased activity of oxidosqualene cyclase resulting from a transferred nucleic acid fragment of the invention. For example, “increased triterpene saponin levels,” may refer to triterpene saponin levels higher than those found in plants of the same species not having the recombinant DNA molecule of the invention comprising a polynucleotide encoding an oxidosqualene cyclase. The “increased triterpene saponin levels” levels may be at least 100 ppm higher, 250 ppm higher, 500 ppm higher, 750 ppm higher, 1000 ppm higher, 1250 ppm higher, 1500 ppm higher, 3000 ppm higher, 6000 ppm higher, or any integer thereof.

The level of triterpene saponins can be determined by measurement of sapongenols. Measurement of sapongenols directly correlates to the level of triterpene saponins. Sapogenols are derived from triterpene saponins via in vitro acid hydrolysis and their measurement provides a relative value which can be directly correlated into the amount of triterpene saponins present in the tissue from which the saponins are extracted.

The triterpene saponin levels can be measured using techniques known in the art. For example, one could use HPLC-MS or HPLC with a light scattering detector(see for example Rupasinghe, H. P. et al, (2003) J. Agri. Food Chem. 51:5888-5894). Alternatively, one could use HPLC with a UV detector (Hubert J, et al. (2005) J. Agric. Food Chem. 53:3923-3930). Other methods include using GC-FAB. (see for example Gee et al. (1993) J Sci Food Agric. 63:201-209). Other methods involve separating saponins using thin layer chromatography (TLC) coupled with densitometry (see for example Oleszek W A. (2002) J. Chromatogr. A 967:147-162.; Gurfinkel D M, and Rao A V (2002) J. Agric. Food Chem. 50:426-430.

It may also be possible to measure triterpene saponins using other methods. For example, methods using various immunoassays (e.g., a radioimmunoassay or ELISA) may be adapted (Wang C C, Prasain J K, and Barnes S. (2002) J. Chromatogr. B Analyt. Technol. Biomed. Life Sci. 777:3-28, Ahamed A et al. (2003) Biochem. Biophys. Res. Commun. 302:587-592).

Total triterpene saponin content varies somewhat by soybean cultivar but is in the range of 0.25% of the seed dry weight (Shiraiwa, M., et al. (1991) Agric. Biol. Chem. 55:323-331).

In accordance with the present invention, a plant includes monocots and dicots. Plants include and are not limited to soybean, alfalfa, peanut, pea, lentil, chick pea, kidney bean, pigeon pea, oat, wheat, and the like. Also within the scope of this invention are seeds or plant parts obtained from such transformed plants. Plant parts include differentiated and undifferentiated tissues, including but not limited to, roots, stems, shoots, leaves, pollen, seeds, grains, tumor tissue, and various forms of cells and culture such as and not limited to single cells, protoplasts, embryos, and callus tissue. The plant tissue may be in plant, organ, tissue, or cell culture.

Preferably, the regenerated plants are self-pollinated to provide homozygous transgenic plants. Pollen obtained from the regenerated plants may also be crossed to plants of agronomically important lines. Conversely, pollen from plants of these agronomically important lines is used to pollinate regenerated plants. A transgenic plant of the present invention, comprising a transferred nucleic acid fragment or recombinant DNA molecule sufficient to increase the levels of triterpene saponins in said transgenic plant is cultivated using methods well known to one skilled in the art.

Seeds from the transformed plant are also part of the invention. Preferentially said seeds are soybean seeds. Also part of the invention are feed and food, including beverages, protein products, and industrial products having increased triterpene saponin levels and prepared from said seeds.

The invention also concerns a method for increasing the triterpene saponin level in a plant comprising: (a) transforming a plant cell with a nucleic acid fragment that increases the activity of at least one oxidosqualene cyclase that catalyzes the cyclization of 2,3-oxidosqualene to form a triterpene to produce a transformed plant cell; (b) growing said transformed plant cell from step (a) under conditions that promote the regeneration of a transgenic plant; and (c) evaluating the transgenic plant of step (b) for an increased level of triterpene saponin when compared to the amount of triterpene saponin in a plant of the same species that is not transformed with said nucleic acid fragment.

The invention also concerns a method for increasing the triterpene saponin level in a plant comprising: (a) creating a recombinant DNA molecule comprising a promoter operably linked to at least one polynucleotide encoding an oxidosqualene cyclase that catalyzes the cyclization of 2,3-oxidosqualene to form a triterpene; (b) transforming a plant cell with said recombinant DNA molecule to produce a transformed plant cell; (c) growing said transformed plant cell from step (b) under conditions that promote the regeneration of a transgenic plant, and (d) evaluating the transgenic plant of step (c) for an increased level of triterpene saponin when compared to the amount of triterpene saponin in a plant of the same species that is not transformed with said recombinant DNA molecule. In a preferred embodiment, the oxidosqualene cyclase catalyzes the cyclization of 2,3-oxidosqualene to form at least one triterpene selected from the group consisting of β-amyrin, lupeol, α-amyrin, isomultiflorenol, thalianol, and any combination thereof. In a more preferred embodiment, the oxidosqualene cyclase is selected from the group consisting of lupeol synthase, β-amyrin synthase, α-amyrin synthase, isomultiflorenol synthase, thalianol synthase and dammarenediol synthase. In an even more preferred embodiment, the oxidosqualene cyclase is a β-amyrin synthase. In an even more preferred embodiment, the β-amyrin synthase has the amino acid sequence of SEQ ID NO:11.

Also included in the invention are transgenic plants or plant parts prepared by the methods of the invention. Also part of the invention are feed and food, including beverages, and industrial products having increased triterpene saponin levels and prepared from transgenic plants or plant parts prepared by the method of the invention.

Soybeans can be boiled or roasted and eaten by themselves, but soybeans, or a part thereof, are most often found as an added ingredient to food or feed.

In another aspect, this invention concerns a protein product high in triterpene saponins obtained from a transformed plant, such as for example a seed or a plant part, described herein. Examples of such product include, but are not limited to, protein isolate, protein concentrate, meal, grits, full fat and defatted flours, textured proteins, textured flours, textured concentrates and textured isolates. In still another aspect, this invention concerns a product high in triterpene saponins extracted from a seed or plant part of a transformed plant described herein. An extracted product may then be used in the production of pills, tablets, capsules or other similar dosage forms.

Methods for obtaining such products are well known to those skilled in the art. For example, in the case of soybean, such products can be obtained in a variety of ways. Conditions typically used to prepare soy protein isolates have been described by (Cho, et al. (1981) U.S. Pat. No. 4,278,597; Goodnight, et al. (1978) U.S. Pat. No. 4,072,670). Soy protein concentrates are produced by three basic processes: acid leaching (at about pH 4.5), extraction with alcohol (about 55-80%), and denaturing the protein with moist heat prior to extraction with water. Conditions typically used to prepare soy protein concentrates have been described (Pass (1975) U.S. Pat. No. 3,897,574 and Campbell et al. (1985) in New Protein Foods, ed. by Altschul and Wilcke, Academic Press, Vol. 5, Chapter 10, Seed Storage Proteins, pp 302-338, among others).

A variety of processed vegetable protein products are produced from plants. Using soybean as a representative example, these range from minimally processed, defatted items such as soybean meal, grits, and flours to more highly processed items such as soy protein concentrates and soy protein isolates. In other soy protein products, such as full-fat soy flour, the oil is not extracted. In addition to these processed products, there are also a number of specialty products based on traditional Oriental processes, which utilize the entire bean as the starting material. Examples include soy milk, soy sauce, tofu, natto, miso, tempeh, and yuba.

Examples of use of soy protein products in human foods include soy protein concentrates, soy protein isolates, textured soy protein, soy milk, and infant formula. Facilities and methods to produce protein concentrates and isolates from soybeans are available across the world.

The protein products of the present invention can be defined as those items produced from seed of a suitable plant, which may be used in feeds and foods, including beverages. For example, soy protein products include and are not limited to those items listed in Table 1.

TABLE 1
Soy Protein Products Derived from Soybean Seeds^{a}

Whole Soybean Products
Roasted Soybeans
Baked Soybeans
Soy Sprouts
Soy Milk
Specialty Soy Foods/Inqredients
Soy Milk
Tofu
Tempeh
Miso
Soy Sauce
Hydrolyzed Vegetable Protein
Whipping Protein
Processed Soy Protein Products
Full Fat and Defatted Flours
Soy Grits
Soy Hypocotyls
Soybean Meal
Soy Protein Isolates
Soy Protein Concentrates
Textured Soy Proteins
Textured Flours and Concentrates
Textured Concentrates
Textured Isolates
Soy Milk
^{a}See Soy Protein Products: Characteristics, Nutritional Aspects and Utilization (1987). Soy Protein Council

Also, within the scope of this invention are food, including beverages, which have incorporated therein a protein product of the invention having high triterpene saponin levels.

The foods to which the protein product of the invention can be incorporated/added include almost all foods, including beverages. For example, there can be mentioned meats such as ground meats, emulsified meats, marinated meats, and meats injected with a high-triterpene product of the invention; beverages such as nutritional beverages, sports beverages, protein fortified beverages, juices, milk, milk alternatives, and weight loss beverages; cheeses such as hard and soft cheeses, cream cheese, and cottage cheese; frozen desserts such as ice cream, ice milk, low fat frozen desserts, and non-dairy frozen desserts; yogurts; soups; puddings; bakery products; and salad dressings; and dips and spreads such as mayonnaise and chip dips. The beverage can be in a liquid or a dry powdered form. The high-triterpene saponin product can be added in an amount selected to deliver a desired dose to the consumer of the food, including beverages.

Still another aspect of this invention concerns a method of producing a high-triterpene product which comprises: (a) cracking the seeds obtained from transformed plants of the invention to remove the meats from the hulls; and (b) flaking the meats obtained in step (a) to obtain the desired flake thickness.

“Processing” refers to any physical and chemical methods used to obtain the products listed in Table 1 and includes, but is not limited to, heat conditioning, flaking and grinding, extrusion, solvent extraction, or aqueous soaking and extraction of whole or partial seeds. Furthermore, “processing” includes the methods used to concentrate and isolate soy protein from whole or partial seeds, as well as the various traditional Oriental methods in preparing fermented soy food products. Trading Standards and Specifications have been established for many of these products (see National Oilseed Processors Association Yearbook and Trading Rules 1991-1992). Products referred to as being “high protein” or “low protein” are those as described by these Standard Specifications. “NSI” refers to the Nitrogen Solubility Index as defined by the American Oil Chemists' Society Method Ac4 41. “KOH Nitrogen Solubility” is an indicator of soybean meal quality and refers to the amount of nitrogen soluble in 0.036 M KOH under the conditions as described by Araba and Dale [(1990) Poult. Sci. 69:76-83]. “White” flakes refer to flaked, dehulled cotyledons that have been defatted and treated with controlled moist heat to have an NSI of about 85 to 90. This term can also refer to a flour with a similar NSI that has been ground to pass through a No. 100 U.S. Standard Screen size. “Cooked” refers to a soy protein product, typically a flour, with an NSI of about 20 to 60. “Toasted” refers to a soy protein product, typically a flour, with an NSI below 20. “Grits” refer to defatted, dehulled cotyledons having a U.S. Standard screen size of between No. 10 and 80. “Soy Protein Concentrates” refer to those products produced from dehulled, defatted soybeans by three basic processes: acid leaching (at about pH 4.5), extraction with alcohol (about 55-80%), and denaturing the protein with moist heat prior to extraction with water. Conditions typically used to prepare soy protein concentrates have been described by Pass [(1975) U.S. Pat. No. 3,897,574; Campbell et al., (1985) in New Protein Foods, ed. by Altschul and Wilcke, Academic Press, Vol. 5, Chapter 10, Seed Storage Proteins, pp 302-338]. “Extrusion” refers to processes whereby material (grits, flour or concentrate) is passed through a jacketed auger using high pressures and temperatures as a means of altering the texture of the material. “Texturing” and “structuring” refer to extrusion processes used to modify the physical characteristics of the material. The characteristics of these processes, including thermoplastic extrusion, have been described previously [Atkinson (1970) U.S. Pat. No. 3,488,770, Horan (1985) In New Protein Foods, ed. by Altschul and Wilcke, Academic Press, Vol. 1A, Chapter 8, pp 367-414]. Moreover, conditions used during extrusion processing of complex foodstuff mixtures that include soy protein products have been described previously [Rokey (1983) Feed Manufacturing Technology III, 222-237; McCulloch, U.S. Pat. No. 4,454,804].

Soybeans are also found in consumer products and in industrial products, as ingredients and intermediates, among others. The web site of the United Soybean Board lists an online Product Guide including the Companies from which they are available. Also, within the scope of this invention are consumer products and industrial products, as well as the ingredients and intermediates, which have incorporated therein a product of the invention having high triterpene saponin levels.

For example, soybean products include and are not limited to those items listed in Table 2.

TABLE 2
Soybean Products Derived from Soybean Seeds

Consumer Products
All purpose lubricants
Animal care products
Auto care products
Building products
Cleaning products
Diesel and gasoline additives
Engine oils for 4 and 2 cycle engines
Candles
Hair care products
Hydraulic fluids
Paint strippers
Air fresheners
Linnen sprays
Truck bed liners
Herbicide adjuvants
Socks
Furniture
Fire logs
Crayons
Oils and lubricants for
bars, chains and sprockets
Ingredients and Intermediates:
Blown soybean oils
Industrial plastics
Industrial proteins
Industrial solvents
Refined and deodorized oils
Surfactants and emulsifiers
Technical grade soybean oils
Unrefined soybean oils
Waxes
Industrial Products
Agricultural adjuvants
Concrete supplies
Dielectric fluids
Dust suppressants
Fuel additives
Hydraulic fluids
Industrial cleaners
Industrial lubricants
Metalworking fluids
Odor reducing materials
Paint strippers
Printing inks
Printing supplies including screenwash
Saw guide oils

### EXAMPLES

The present invention is further defined in the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

The disclosure of each reference set forth herein is incorporated herein by reference in its entirety.

### Example 1

### Preparation of Chimeric Oxidosqualene Cyclase Plasmid

A recombinant DNA molecule designed to overexpress β-amyrin synthase was transformed into soybean embryogenic suspension culture cells to test the ability to increase triterpene saponin production. Transformed cells were regenerated into plants and the triterpene saponin measured. The recombinant DNA molecule designed to overexpress β-amyrin synthase was named PHP20767 and was prepared by inserting nucleotides encoding a β-amyrin synthase into the unique Not I restriction endonuclease site of plasmid pDN10. Preparation of plasmids pDN10 and PHP20767 follows.

A. Construction of Plasmid pDN10

Plasmid pDN10 is an intermediate cloning vector comprising a bacterial origin of replication, bacterial and plant selectable marker gene expression cassettes, and a promoter and terminator separated by a unique Not I restriction endonuclease site. Plasmid pDN10 was prepared by ligating a fragment comprising a plant selectable marker gene expression cassette and a cassette comprising a promoter and terminator separated by a unique Not I restriction endonuclease site to a fragment comprising the bacterial origin of replication and a bacterial selectable marker gene. Preparation of these two fragments follows.

The fragment comprising a plant selectable marker gene expression cassette and a cassette comprising a promoter and terminator separated by a unique Not I restriction endonuclease site has 6383 bp and was obtained by Kpn I digestion of plasmid pKS231. Plasmid pKS231 has been deposited with the American Type Culture Collection (ATCC), 10801 University Boulevard, Manassas, Va., and bears ATCC Accession Number PTA-6148. The nucleotide sequence of the 6383 bp fragment is shown in SEQ ID NO:1. This fragment contains two cassettes: 1) a plant selectable marker gene cassette, and 2) a cassette comprising a promoter and terminator separated by a unique Not I restriction endonuclease site. The plant selectable marker gene expression cassette comprises a 1.3-Kb DNA fragment that functions as the promoter for a soybean S-adenosylmethionine synthase (SAMS) gene directing expression of a mutant soybean acetolactate synthase (ALS) gene which is followed by the soybean ALS 3′ transcription terminator. The 1.3-Kb DNA fragment that functions as the promoter for a soybean SAMS gene has been described in PCT Publication No. WO 00/37662, published Jun. 29, 2000. The mutant soybean ALS gene encodes an enzyme that is resistant to inhibitors of ALS, such as sulfonylurea herbicides.

Mutant plant ALS genes encoding enzymes resistant to sulfonylurea herbicides are described in U.S. Pat. No. 5,013,659. One such mutant is the tobacco SURB-Hra gene, which encodes an herbicide-resistant ALS with two substitutions in the amino acid sequence of the protein. This tobacco herbicide-resistant ALS contains alanine instead of proline at position 191 in the conserved “subsequence B” and leucine instead of tryptophan at position 568 in the conserved “subsequence F” (U.S. Pat. No. 5,013,659; Lee et al., 1988, EMBO J. 7:1241-1248).

The mutant soybean ALS gene was constructed using a polynucleotide for a soybean ALS to which the two Hra-like mutations were introduced by site directed mutagenesis. Thus, this recombinant DNA fragment will translate to a soybean ALS having alanine instead of proline at position 183 and leucine instead of tryptophan at position 560. The deduced amino acid sequence of the mutant soybean ALS present in the mutant ALS gene is shown in SEQ ID NO:2. During construction of SAMS promoter-mutant ALS expression cassette, the coding region of the soybean ALS gene was extended at the 5′ end by five additional codons, resulting in five amino acids, added to the amino-terminus of the ALS protein (amino acids 1 through 5 of SEQ ID NO:2). These extra amino acids are adjacent to and presumably removed with the transit peptide during targeting of the mutant soybean ALS protein to the plastid.

The cassette comprising a promoter and terminator separated by a unique Not I restriction endonuclease site comprises the Kunitz trypsin inhibitor (KTi3) promoter, a unique Not I restriction endonuclease site, and the KTi3 terminator region. This cassette comprises about 2088 nucleotides of the KTi3 promoter, a unique Not I restriction endonuclease site, and about 202 nucleotides of the KTi3 transcription terminator. The gene encoding KTi3 has been described (Jofuku, K. D. and Goldberg, R. B., 1989, Plant Cell 1:1079-1093).

The fragment comprising the bacterial origin of replication and bacterial selectable marker gene was obtained by PCR amplification from plasmid pKS210. Plasmid pKS210 is derived from the commercially available cloning vector pSP72 (Promega, Madison, Wis.). In plasmid pKS210 the beta lactamase coding region in vector pSP72 has been removed and a hygromycin phosphotransferase (HPT) coding region has been added under the control of the T7 promoter and terminator for use as a selectable marker in E. coli. The nucleotide sequence of plasmid pKS210 is shown in SEQ ID NO:3. A fragment of plasmid pKS210, comprising the bacterial origin of replication and the HPT gene, was amplified by PCR using primers BM5 and BM6, and plasmid pKS210 as a template, with Advantage High Fidelity polymerase (BD Biosciences, San Jose, Calif.) according to the manufacturer's instructions. Oligonucleotide primers BM5 and BM6 have the nucleotide sequences set forth in SEQ ID NO:4 and SEQ ID NO:5, respectively, and have the sequences set forth as follows:

| | |
|---|---|
| 5′-GCCGGGGTACCGGCGCGCCCGATCATCCGGATATAG | SEQ ID NO:4 |
| TTCC-3′ | |
| 5′-GCCGGGGTACCGGCGCGCCGTTCTATAGTGTCACCT | SEQ ID NO:5 |
| AATC-3′ | |

Amplification was performed using a GeneAmp PCR System 9700 machine (Applied Biosystems, Foster City, Calif.) and the resulting 2600 bp fragment was gel purified using the Qiagen Gel Purification System (Qiagen Inc., Valencia, Calif.), digested with Kpn I, and treated with Calf Intestinal Alkaline Phosphatase.

The two Kpn I fragments described above, were ligated together and transformed into E. coli. Bacterial colonies were selected and grown overnight in LB media with appropriate antibiotic selection. DNA was isolated from the resulting culture using a Qiagen Miniprep Kit according to the manufacturer's protocol and then analyzed by restriction digest. The resulting plasmid was named pDN10 and its nucleotide sequence is shown in SEQ ID NO:6.

Construction of Plasmid PHP20767

Plasmid PHP20767 was prepared by inserting a polynucleotide fragment encoding a β-amyrin synthase into plasmid pDN10 as follows.

A polynucleotide fragment encoding a β-amyrin synthase was obtained by PCR amplification from clone src3c.pk024.m11. The cDNA insert in clone src3c.pk024.m11 has been previously identified as encoding a β-amyrin synthase due to its demonstrated ability of producing β-amyrin (PCT publication No. WO01/66773, published 13 Sep. 2001). The nucleotide sequence of the cDNA insert in clone src3c.pk024.m11 is shown in SEQ ID NO:7. The coding portion of the cDNA insert in clone src3c.pk024.m11 was amplified using oligonucleotide primers BM7 and BM8 and Advantage High Fidelity polymerase. Oligonucleotide primers BM7 and BM8 have the nucleotide sequences set forth in SEQ ID NO:8 and SEQ ID NO:9, respectively, and have the sequences set forth as follows:

| | |
|---|---|
| 5′-GCGGCCGCATGTGGAGGCTGAAGATAGCAG-3′ | SEQ ID NO:8 |
| 5′-GCGGCCGCTTAAACTTCAGTGGAAGGCAATG-3′ | SEQ ID NO:9 |

The resulting amplification product was introduced into plasmid pCR2.1 using the TOPO TA Cloning Kit (Invitrogen, Carlsbad, Calif.) and its nucleotide sequence is shown in SEQ ID NO:10. The resulting clone was completely sequenced using a mixture of external and internal primers and shown to have the expected sequence.

The amplified product having the nucleotide sequence shown in SEQ ID NO:10 was removed from plasmid pCR2.1 by digestion with the restriction endonuclease Not I. Plasmid pDN10 was digested with Not I and treated with Calf Intestinal Alkaline Phosphatase according to the manufacturer's instructions. The 8911 bp fragment from clone pDN10 and the amplified product having the nucleotide sequence shown in SEQ ID NO:10 were gel purified using Qiagen gel purification kit. These two fragments were ligated together and transformed into E. coli. Bacterial colonies were selected and grown overnight in LB media with appropriate antibiotic selection. Plasmid DNA was isolated from the resulting cultures using Qiagen Miniprep Kit according to the manufacturer's protocol and then analyzed by restriction digest. A plasmid DNA with the appropriate restriction pattern was named PHP20767, its nucleotide sequence is shown in SEQ ID NO:12, and it was used for transformation of somatic soybean embryo cultures as described in Example 2 below.

### Example 2

### Transformation of Somatic Soybean (Glycine max) Embryo Cultures and Regeneration of Soybean Plants

Soybean embryogenic suspension cultures were transformed by the method of particle gun bombardment using procedures known in the art (Klein et al. (1987) Nature (London) 327:70-73; U.S. Pat. No. 4,945,050; Hazel, et al. (1998) Plant Cell. Rep. 17:765-772; Samoylov, et al. (1998) In Vitro Cell Dev. Biol.-Plant 34:8-13). In particle gun bombardment procedures it is possible to use purified entire plasmid DNA or purified DNA fragments containing only the recombinant DNA expression cassette(s) of interest.

For transformation using PHP20767, the recombinant DNA fragment was isolated from the entire plasmid by Asc I digestion and was purified by gel electrophoresis before being used for bombardment. For every eight bombardment transformations, 30 μL of solution were prepared with 3 mg of 0.6 mm gold particles and 1 to 90 picograms (pg) of DNA fragment per base pair of DNA fragment. The DNA/particle suspension was sonicated three times for one second each. Five microliters of the DNA-coated gold particles were then loaded on each macro carrier disk.

Stock tissue for these transformation experiments were obtained by initiation from soybean immature seeds. Secondary embryos were excised from explants after 6 to 8 weeks on culture initiation medium. The initiation medium was an agar-solidified modified MS medium (Murashige and Skoog (1962) Physiol. Plant. 15:473-497) supplemented with vitamins, 2,4-D, and glucose. Secondary embryos were placed in flasks in liquid culture maintenance medium and maintained for 7-9 days on a gyratory shaker at 26±2° C. under ˜80 μEm⁻²s⁻¹ light intensity. The culture maintenance medium was a modified MS medium supplemented with vitamins, 2,4-D, sucrose, and asparagine. Prior to bombardment, clumps of tissue were removed from the flasks and moved to an empty 60×15 mm petri dish for bombardment. Tissue was dried by blotting on Whatman #2 filter paper. Approximately 100-200 mg of tissue corresponding to 10-20 clumps (1-5 mm in size each) was used per plate of bombarded tissue.

After bombardment, tissue from each bombarded plate was divided and placed into two flasks of liquid culture maintenance medium per plate of bombarded tissue. For transformation experiments using the Asc I fragment from PHP20767, seven days post bombardment, the liquid medium in each flask was replaced with fresh culture maintenance medium supplemented with 100 ng/mL selective agent (selection medium). For selection of transformed soybean cells the selective agent used was a sulfonylurea (SU) compound with the chemical name, 2-chloro-N-((4-methoxy-6 methy-1,3,5-triazine-2-yl)aminocarbonyl)benzenesulfonamide (common names: DPX-W4189 and chlorsulfuron). Chlorsulfuron is the active ingredient in the DuPont sulfonylurea herbicide, GLEAN®. The selection medium containing SU was replaced every week for 6-8 weeks. After the 6-8 week selection period, islands of green, transformed tissue were observed growing from untransformed, necrotic embryogenic clusters. These putative transgenic events were isolated and kept in media with SU at 100 ng/mL for another 2-6 weeks with media changes every 1-2 weeks to generate new, clonally propagated, transformed embryogenic suspension cultures. Embryos spent a total of around 8-12 weeks in SU.

Suspension cultures were subcultured and maintained as clusters of immature embryos and also regenerated into whole plants by maturation and germination of individual somatic embryos. Maturation and germination of individual subcultured somatic embryos resulted in regenerated whole plants. Plants were grown in a greenhouse and allowed to produce seed, which was analyzed for their soyasapogenols content as an indication of the amount of triterpene saponins present.

### Example 3

### Analyses of Sovasapogenols in Transgenic Soybean Plants

The effect of the overexpression of β-amyrin synthase on the triterpene saponin content in soybean plants was measured by analyzing the T1 and T2 seed obtained from soybean transgenic plants transformed with plasmid PHP20767 as explained in Example 2, above. Soyasapogenol A and soyasapogenol B content was calculated after removing the sugar moieties from triterpene saponins by acid hydrolysis and comparing the results to concentration curves prepared by High Performance Liquid Chromatography/Mass Spectrometry (LC/MS) of authentic standards. Because soyasapogenol A and soyasapogenol B are derived from triterpene saponins, their measurement provides a relative value for the amount of triterpene saponins present. Transgenic soybean plants were analyzed as follows.

Five to ten seeds per transformant were combined and whole soybeans pulverized to a fine powder using Geno/Grinder™ Model 2000 (SPEX Certiprep, Metuchen, N.J.). About 100 mg ground soybean was accurately weighed into a micro centrifuge tube with screw cap, and a ¼ inch stainless steel ball was added along with 1 mL of 60% acetonitrile in water. The mixture was agitated on a Geno/Grinder™ for 1 minute with the machine set at 1500 strokes per minute and then placed on an end-over-end tumbler for 1 hour. The tube was then returned to the Geno/Grinder™ for 1 minute with the machine set at 1500 strokes per minute. Samples were centrifuged at 12,000 rpm for 4 minutes and then the supernatant transferred to a 13×100 mm glass test tube fitted with a Teflon®-lined cap. The extraction procedure was repeated once and the supernatants combined into the same 13×100 mm glass test tube. To the tube containing the combined supernatants, 0.1 mL of 12N HCl was added. After mixing, the tube was placed into an 80° C. heating block overnight (16 to 17 hours).

After overnight incubation, the tube was removed from the heating block and allowed to cool to room temperature. Next, 5.0 mL of 12.5% methanol in acetonitrile were added to the extracts and mixed. The extract volume was measured and recorded. The tubes were centrifuged for 10 minutes at 3500 rpm at 20° C. and an aliquot of the supernatant was placed into an HPLC vial, along with an equal volume of 12.5% methanol in acetonitrile, to analyze the soyasapogenols using LC/MS.

LC/MS was performed using a Waters™ (Waters Corp., Milford, Mass.) 2690 Alliance HPLC interfaced with a ThermoFinnigan (San Jose, Calif.) LCQ™ mass spectrometer. Samples were maintained at 20° C. prior to injection. A 10 μL sample was injected onto a Phenomenex® (Torrance, Calif.) Luna™ C18 column (3 μ, 4.6 mm×50 mm), equipped with a guard cartridge of the same material, and maintained at 40° C. Compounds were eluted from the column at a flow rate of 0.8 mL/minute using a solvent gradient. For the first two minutes the eluent was a 50/50 mixture of solvent A (0.1% formic acid in water) and solvent B (0.1% formic acid in acetonitrile). From 2 to 5 minutes the eluent was a linear gradient from 50% solvent B to 100% solvent B. From 5 to 8 minutes the eluent was 100% solvent B, and from 8 to 11 minutes the eluent was a 50/50 mixture of solvent A and solvent B. The mass spectrometer was equipped with an atmospheric pressure chemical ionization (APCI) probe set to scan m/z of 250 to 500 in positive ion mode. The vaporizer temperature was set to 400° C., the capillary temperature was at 160° C., and the sheath gas flow was at 60 psi. Identification and quantification of soyasapogenol A and B was based on m/z and co-chromatography of authentic standards (Apin Chemicals, LTD, Oxon, UK or ChromaDex, Santa Ana, Calif.).

Triterpene Saponin Levels of Plants Transformed with PHP20676

The levels of soyasapogenol in 102 plants representing 55 independent events containing pHP20676 are shown in Table 3. Table 3 presents the total level of soyasapogenols (in ppm) obtained for seeds from these plants.

TABLE 3

| Plant | Soyasapogenols (ppm) |
|---|---|
| 3786.3.1.1 | 3381 |
| 3786.3.1.2 | 2466 |
| 3786.4.1.2 | 2000 |
| 3786.5.1.1 | 3045 |
| 3786.5.1.2 | 3373 |
| 3786.6.2.1 | 3039 |
| 3786.6.2.2 | 3251 |
| 3786.7.5.1 | 2998 |
| 3786.7.5.2 | 3290 |
| 3786.7.6.1 | 3136 |
| 3786.7.6.2 | 3398 |
| 3786.7.7.1 | 2237 |
| 3786.7.7.2 | 2522 |
| 3792.1.3.1 | 2964 |
| 3792.1.3.2 | 1988 |
| 3792.1.6.1 | 3548 |
| 3792.1.6.2 | 3434 |
| 3786.2.1.1 | 3050 |
| 3786.2.1.2 | 3780 |
| 3786.2.2.1 | 3183 |
| 3786.2.2.2 | 3502 |
| 3786.3.3.1 | 3408 |
| 3786.3.3.2 | 3534 |
| 3786.4.2.1 | 1259 |
| 3786.4.2.2 | 1542 |
| 3786.5.5.1 | 4654 |
| 3786.5.5.2 | 4037 |
| 3786.5.6.1 | 2435 |
| 3786.7.2.1 | 4234 |
| 3786.7.2.2 | 4387 |
| 3786.7.4.1 | 2839 |
| 3786.7.4.2 | 3345 |
| 3786.8.2.1 | 3732 |
| 3786.8.2.2 | 3900 |
| 3788.1.1.1 | 4355 |
| 3788.1.3.1 | 1558 |
| 3788.1.3.2 | 1994 |
| 3788.1.4.1 | 3966 |
| 3788.1.4.2 | 3905 |
| 3788.1.7.1 | 3506 |
| 3788.1.7.2 | 3817 |
| 3788.3.1.1 | 4584 |
| 3788.3.1.2 | 4310 |
| 3788.3.2.1 | 604 |
| 3788.3.2.2 | 716 |
| 3788.4.3.2 | 3319 |
| 3791.6.1.1 | 2412 |
| 3791.6.1.2 | 2520 |
| 3792.1.1.1 | 3285 |
| 3792.1.1.2 | 2778 |
| 3792.1.2.1 | 183 |
| 3792.1.2.2 | 969 |
| 3792.1.5.1 | 640 |
| 3792.1.5.2 | 990 |
| 3792.1.8.1 | 890 |
| 3792.1.8.2 | 900 |
| 3792.1.11.2 | 2612 |
| 3792.2.1.1 | 2150 |
| 3792.2.1.2 | 2696 |
| 3792.5.1.1 | 294 |
| 3792.5.1.2 | 299 |
| 3792.5.2.1 | 2942 |
| 3792.5.2.2 | 3059 |
| 3792.5.4.1 | 3212 |
| 3792.5.4.2 | 3143 |
| 3792.5.5.1 | 435 |
| 3792.5.5.2 | 1090 |
| 3792.5.6.1 | 2553 |
| 3792.5.6.2 | 2284 |
| 3792.7.1.1 | 3160 |
| 3792.7.1.2 | 1429 |
| 3792.7.2.1 | 544 |
| 3792.7.2.2 | 631 |
| 3786.6.3.2 | 2526 |
| 3791.4.1.1 | 3576 |
| 3791.4.1.2 | 3180 |
| 3792.3.2.1 | 1130 |
| 3792.3.2.2 | 2548 |
| 3786.5.7.1 | 5303 |
| 3786.5.7.2 | 6104 |
| 3786.8.4.2 | 4106 |
| 3786.8.5.1 | 4841 |
| 3786.8.5.2 | 5018 |
| 3786.8.6.1 | 5096 |
| 3786.8.6.2 | 2054 |
| 3788.1.2.1 | 5540 |
| 3788.1.2.2 | 3815 |
| 3788.1.8.1 | 3795 |
| 3788.1.8.2 | 3908 |
| 3788.2.1.1 | 3519 |
| 3788.4.1.1 | 3429 |
| 3788.4.2.1 | 4216 |
| 3788.4.2.2 | 4005 |
| 3788.4.4.1 | 3310 |
| 3788.4.4.2 | 3242 |
| 3788.4.5.1 | 3999 |
| 3788.4.5.2 | 3769 |
| 3792.3.3.1 | 3876 |
| 3792.3.3.2 | 3719 |
| 3792.5.7.1 | 4139 |
| 3792.5.7.2 | 3863 |
| 3792.1.4.1 | 2705 |

Levels of soyasapogenols vary by soybean variety. The mean soyasapogenol level for seed from 12 non-transformed Jack plants regenerated from plant tissue put through the tissue culture process but not through the bombardment process was 3485±592 ppm. Soyasapogenol levels in transgenic plants comprising pHP20676 varied between 183 and 6104 ppm. These results indicate that overexpression of β-amyrin synthase can result in an increase of triterpene saponins.

The levels of soyasapogenol of T2 seeds from plants representing 3 independent events containing pHP20676 are shown in Table 4. Table 4 presents the levels of soyasapogenol A and B and the total level of soyasapogenols (in ppm) obtained for seeds from these plants. The seeds were analyzed as described above. The soyasapogenol level for transgenic T2 seeds ranged between 3391 and 5938 ppm. These results further support that over expression of β-amyrin synthase can result in an increase of triterpene saponins.

TABLE 4

| | Soyasapogenol A | Soyasapogenol B | Soyasapogenol A & |
|---|---|---|---|
| Plant number | (ppm) | (ppm) | B (ppm) |
| 3786.5.7.1 | 1097 | 3100 | 4196 |
| 3786.5.7.1 | 1609 | 4327 | 5936 |
| 3786.5.7.1 | 1381 | 3515 | 4896 |
| 3786.5.7.1 | 1126 | 3577 | 4704 |
| 3786.5.7.1 | 1291 | 3501 | 4791 |
| 3786.5.7.1 | 1074 | 2678 | 3752 |
| 3786.5.7.1 | 1764 | 4468 | 6232 |
| 3786.5.7.1 | 1015 | 3167 | 4182 |
| 3786.5.7.1 | 1414 | 3941 | 5355 |
| 3786.5.7.1 | 1053 | 3029 | 4082 |
| 3786.5.7.1 | 823 | 3329 | 4152 |
| 3786.5.7.1 | 1165 | 4052 | 5217 |
| 3786.5.7.2 | 1206 | 3701 | 4907 |
| 3786.5.7.2 | 872 | 2519 | 3391 |
| 3786.5.7.2 | 926 | 3124 | 4050 |
| 3786.5.7.2 | 1045 | 3135 | 4180 |
| 3786.5.7.2 | 912 | 3096 | 4008 |
| 3786.5.7.2 | 1209 | 3461 | 4671 |
| 3786.5.7.2 | 956 | 3094 | 4050 |
| 3786.5.7.2 | 1342 | 3219 | 4561 |
| 3786.5.7.2 | 941 | 4430 | 5371 |
| 3786.5.7.2 | 1061 | 3494 | 4556 |
| 3786.5.7.2 | 1031 | 4529 | 5560 |
| 3786.5.7.2 | 1038 | 3766 | 4805 |
| 3786.8.5.1 | 1066 | 3373 | 4438 |
| 3786.8.5.1 | 945 | 3188 | 4133 |
| 3786.8.5.1 | 709 | 3381 | 4090 |
| 3786.8.5.1 | 729 | 3638 | 4367 |
| 3786.8.5.1 | 1185 | 3676 | 4861 |
| 3786.8.5.1 | 808 | 3048 | 3857 |
| 3786.8.5.1 | 1178 | 3339 | 4517 |
| 3786.8.5.2 | 1066 | 3626 | 4692 |
| 3786.8.5.2 | 738 | 2848 | 3586 |
| 3786.8.5.2 | 867 | 3234 | 4101 |
| 3786.8.5.2 | 735 | 2842 | 3576 |
| 3788.1.2.1 | 898 | 3501 | 4399 |
| 3788.1.2.1 | 997 | 3086 | 4083 |
| 3788.1.2.1 | 887 | 2829 | 3716 |
| 3788.1.2.1 | 758 | 2811 | 3570 |
| 3788.1.2.1 | 697 | 2913 | 3609 |
| 3788.1.2.1 | 1112 | 3406 | 4518 |

What is claimed is:

1. A transformed plant with increased levels of triterpene saponins, comprising a nucleic acid fragment that increases the activity of at least one oxidosqualene cyclase that catalyzes the cyclization of 2,3-oxidosqualene to form a triterpene, wherein the increased activity of the oxidosqualene cyclase is sufficient to increase triterpene saponin levels in the plant.

2. A plant transformed with at least one recombinant DNA molecule comprising a promoter operably linked to at least one polynucleotide encoding an oxidosqualene cyclase, said recombinant DNA molecule sufficient to increase triterpene saponin levels; or any progeny of said plant, wherein said progeny comprise said recombinant DNA molecule.

3. The plant of claim 1 wherein said oxidosqualene cyclase catalyzes cyclization of 2,3-oxidosqualene to form a triterpene selected from the group consisting of β-amyrin, lupeol, α-amyrin, isomultiflorenol, thalianol, and any combination thereof.

4. The plant of claim 1 wherein said oxidosqualene cyclase is β-amyrin synthase.

5. The plant of claim 1 wherein said promoter is selected from the group consisting of a seed-specific promoter, a root-specific promoter, a vacuole-specific promoter, a leaf-specific promoter, a pod-specific promoter, and an embryo-specific promoter.

6. The plant of claim 1 selected from the group consisting of soybean, alfalfa, peanut, pea, lentil, chickpea, kidney bean, pigeon pea, oat, and wheat.

7. A seed derived from the plant of claim 1.

8. A seed derived from the plant of claim 1 wherein said plant is a soybean.

9. A protein product prepared from the seed of claim 7.

10. A protein product prepared from the seed of claim 8.

11. Feed prepared from the seed of claim 7.

12. Feed prepared from the seed of claim 8.

13. A food prepared from the seed of claim 7.

14. A food prepared from the seed of claim 8.

15. An industrial product prepared from the seed of claim 7.

16. An industrial product prepared from the seed of claim 8.

17. A method for increasing triterpene saponin level in a plant comprising:
(a) creating a recombinant DNA molecule comprising a promoter operably linked to at least one polynucleotide encoding an oxidosqualene cyclase;
(b) transforming a plant cell with said recombinant DNA molecule to produce a transformed plant cell;
(c) growing said transformed plant cell from step (b) under conditions that promote the regeneration of a transgenic plant, and
(d) evaluating the transgenic plant of step (c) for an increased level of triterpene saponin when compared to the amount of triterpene saponin in a plant of the same species that is not transformed with said recombinant DNA molecule.

18. The method of claim 17 wherein said oxidosqualene cyclase catalyzes the cyclization of 2,3 oxidosqualene to form a triterpene selected from the group consisting of β-amyrin, lupeol, α-amyrin, isomultiflorenol, thalianol, and any combination thereof.

19. The method of claim 17 wherein said oxidosqualene cyclase is β-amyrin synthase.

20. The method of claim 17 wherein said promoter is selected from the group consisting of a seed-specific promoter, a root-specific promoter, a vacuole-specific promoter, a leaf-specific promoter, a pod-specific promoter, and an embryo-specific promoter.

21. The method of claim 17 wherein said plant is selected from the group consisting of soybean, alfalfa, peanut, pea, lentil, chick pea, pigeon pea, oat, and wheat.

22. A transgenic plant or plant part prepared by the method of claim 17.

23. A seed derived from the transgenic plant prepared by the method of claim 17.

24. A seed derived from the transgenic plant prepared by the method of claim 17 wherein said plant is a soybean.

25. A product prepared from the seed of claim 23.

26. A product prepared from the seed of claim 24.

27. Feed prepared from the seed of claim 23.

28. Feed prepared from the seed of claim 24.

29. A food prepared from the seed of claim 23.

30. A food prepared from the seed of claim 24.

31. An industrial product prepared from the seed of claim 23.

32. An industrial product prepared from the seed of claim 24.

33. A plant transformed with a recombinant DNA molecule comprising a seed specific promoter operably linked to a DNA fragment encoding a β-amyrin synthase and having a nucleotide sequence of SEQ ID NO:10, said recombinant DNA molecule sufficient to increase production of a triterpene saponin; or any progeny of said plant wherein said progeny comprise said recombinant DNA molecule.

34. A method for increasing the triterpene saponin level in a transgenic soybean plant comprising:
(a) creating a recombinant DNA molecule comprising a seed specific promoter operably linked to a DNA fragment encoding a β-amyrin synthase and having a nucleotide sequence of SEQ ID NO:10;
(b) transforming a soybean plant cell with said recombinant DNA molecule to produce a transformed plant cell;
(c) growing said transformed plant cell from step (b) under conditions that promote the regeneration of a transgenic plant, and
(d) evaluating the transgenic plant of step (c) for an amount of saponin that is increased when compared to the amount of triterpene saponin in a soybean plant that is not transformed with said recombinant DNA molecule.

35. A method for increasing the triterpene saponin level in a plant comprising:
(a) transforming a plant cell with a nucleic acid molecule that increases the activity of at least one oxidosqualene cyclase that catalyzes the cyclization of 2,3-oxidosqualene to form a triterpene to produce a transformed plant cell;
(b) growing said transformed plant cell from step (a) under conditions that promote the regeneration of a transgenic plant; and
(c) evaluating the transgenic plant of step (b) for an increased level of triterpene saponin when compared to the amount of triterpene saponin in a plant of the same species that is not transformed with said nucleic acid molecule.


SEQUENCE LISTING (12 sequences)

SEQ ID NO: 1 - DNA, 6383 bp, Artificial
  Kpn I fragment from pKS231
  misc_feature (1212)..(1212): n is a, c, g, or t
cgggccccccctcgaggtcgacggtatcgataagcttgatatcgaattcctgcagcccgggggatccactagttctagagcggcccgcgccgtcgacggatataatgagccgtaaacaaagatgattaagtagtaattaatacgtactagtaaaagtggcaaaagataacgagaaagaaccaatttctttgcattcggccttagcggaaggcatatataagctttgattattttatttagtgtaatgatttcgtacaaccaaagcatttatttagtactctcacacttgtgtcgcggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaagtgtgttattaacccttctcttcgaggatccaagcttggcgcgggccgccaccgcggtggggtcgactctagtaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgttggactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataaccatgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatatcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccgggtac

SEQ ID NO: 2 - protein, 656 aa, Artificial Sequence
  Mutant soybean ALS
MPHNTMAATASRTTRFSSSSSHPTFPKRITRSTLPLSHQTLTKPNHALKIKCSISKPPTAAPFTKEAPTTEPFVSRFASGEPRKGADILVEALERQGVTTVFAYPGGASMEIHQALTRSAAIRNVLPRHEQGGVFAAEGYARSSGLPGVCIATSGPGATNLVSGLADALMDSVPVVAITGQVARRMIGTDAFQETPIVEVSRSITKHNYLILDVDDIPRVVAEAFFVATSGRPGPVLIDIPKDVQQQLAVPNWDEPVNLPGYLARLPRPPAEAQLEHIVRLIMEAQKPVLYVGGGSLNSSAELRRFVELTGIPVASTLMGLGTFPIGDEYSLQMLGMHGTVYANYAVDNSDLLLAFGVRFDDRVTGKLEAFASRAKIVHIDIDSAEIGKNKQAHVSVCADLKLALKGINMILEEKGVEGKFDLGGWREEINVQKHKFPLGYKTFQDAISPQHAIEVLDELTNGDAIVSTGVGQHQMWAAQFYKYKRPRQWLTSGGLGAMGFGLPAAIGAAVANPGAVVVDIDGDGSFIMNVQELATIRVENLPVKILLLNNQHLGMVVQLEDRFYKSNRAHTYLGDPSSESEIFPNMLKFADACGIPAARVTKKEELRAAIQRMLDTPGPYLLDVIVPHQEHVLPMIPSNGSFKDVITEGDGRTRY

SEQ ID NO: 3 - DNA, 8966 bp, Artificial Sequence
  Plasmid pKS210
  misc_feature (1177)..(1177): n is a, c, g, or t
gatcctcgaagagaagggttaataacacattttttaacatttttaacacaaattttagttatttaaaaatttattaaaaaatttaaaataagaagaggaactctttaaataaatctaacttacaaaatttatgatttttaataagttttcaccaataaaaaatgtcataaaaatatgttaaaaagtatattatcaatattctctttatgataaataaaaagaaaaaaaaaataaaagttaagtgaaaatgagattgaagtgactttaggtgtgtataaatatatcaaccccgccaacaatttatttaatccaaatatattgaagtatattattccatagcctttatttatttatatatttattatataaaagctttatttgttctaggttgttcatgaaatatttttttggttttatctccgttgtaagaaaatcatgtgctttgtgtcgccactcactattgcagctttttcatgcattggtcagattgacggttgattgtatttttgttttttatggttttgtgttatgacttaagtcttcatctctttatctcttcatcaggtttgatggttacctaatatggtccatgggtacatgcatggttaaattaggtggccaactttgttgtgaacgatagaattttttttatattaagtaaactatttttatattatgaaataataataaaaaaaatattttatcattattaacaaaatcatattagttaatttgttaactctataataaaagaaatactgtaacattcacattacatggtaacatctttccaccctttcatttgttttttgtttgatgactttttttcttgtttaaatttatttcccttcttttaaatttggaatacattatcatcatatataaactaaaatactaaaaacaggattacacaaatgataaataataacacaaatatttataaatctagctgcaatatatttaaactagctatatcgatattgtaaaataaaactagctgcattgatactgataaaaaaatatcatgtgctttctggactgatgatgcagtatacttttgacattgcctttattttatttttcagaaaagctttcttagttctgggttcttcattatttgtttcccatctccattgtgaattgaatcatttgcttcgtgtcacaaatacaatttagntaggtacatgcattggtcagattcacggtttattatgtcatgacttaagttcatggtagtacattacctgccacgcatgcattatattggttagatttgataggcaaatttggttgtcaacaatataaatataaataatgtttttatattacgaaataacagtgatcaaaacaaacagttttatctttattaacaagattttgtttttgtttgatgacgttttttaatgtttacgctttcccccttcttttgaatttagaacactttatcatcataaaatcaaatactaaaaaaattacatatttcataaataataacacaaatatttttaaaaaatctgaaataataatgaacaatattacatattatcacgaaaattcattaataaaaatattatataaataaaatgtaatagtagttatatgtaggaaaaaagtactgcacgcataatatatacaaaaagattaaaatgaactattataaataataacactaaattaatggtgaatcatatcaaaataatgaaaaagtaaataaaatttgtaattaacttctatatgtattacacacacaaataataaataatagtaaaaaaaattatgataaatatttaccatctcataagatatttaaaataatgataaaaatatagattattttttatgcaactagctagccaaaaagagaacacgggtatatataaaaagagtacctttaaattctactgtacttcctttattcctgacgtttttatatcaagtggacatacgtgaagattttaattatcagtctaaatatttcattagcacttaatacttttctgttttattcctatcctataagtagtcccgattctcccaacattgcttattcacacaactaactaagaaagtcttccatagccccccaagcggccggagctggtcatctcgctcatcgtcgagtcggcggccggagctggtcatctcgctcatcgtcgagtcggcggccgccgactcgacgatgagcgagatgaccagctccggccgccgactcgacgatgagcgagatgaccagctccggccgcgacacaagtgtgagagtactaaataaatgctttggttgtacgaaatcattacactaaataaaataatcaaagcttatatatgccttccgctaaggccgaatgcaaagaaattggttctttctcgttatcttttgccacttttactagtacgtattaattactacttaatcatctttgtttacggctcattatatccgtcgacggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccaagcttggatccgcgccaagcttggatcctagaactagaaacgtgatgccacttgttattgaagtcgattacagcatctattctgttttactatttataactttgccatttctgacttttgaaaactatctctggatttcggtatcgctttgtgaagatcgagcaaaagagacgttttgtggacgcaatggtccaaatccgttctacatgaacaaattggtcacaatttccactaaaagtaaataaatggcaagttaaaaaaggaatatgcattttactgattgcctaggtgagctccaagagaagttgaatctacacgtctaccaaccgctaaaaaaagaaaaacattgatatgtaacctgattccattagcttttgacttcttcaacagattctctacttagatttctaacagaaatattattactagcacatcattttcagtctcactacagcaaaaaatccaacggcacaatacagacaacaggagatatcagactacagagatagatagatgctactgcatgtagtaagttaaataaaaggaaaataaaatgtcttgctaccaaaactactacagactatgatgctcaccacaggccaaatcctgcaactaggacagcattatcttatatatattgtacaaaacaagcatcaaggaacatttggtctaggcaatcagtacctcgttctaccatcaccctcagttatcacatccttgaaggatccattactgggaatcatcggcaacacatgctcctgatggggcacaatgacatcaagaaggtaggggccaggggtgtccaacattctctgaattgccgctctaagctcttccttcttcgtcactcgcgctgccggtatcccacaagcatcagcaaacttgagcatgtttgggaatatctcgctctcgctagacggatctccaagataggtgtgagctctattggacttgtagaacctatcctccaactgaaccaccatacccaaatgctgattgttcaacaacaatatcttaactgggagattctccactcttatagtggccaactcctgaacattcatgatgaaactaccatccccatcaatgtcaaccacaacagccccagggttagcaacagcagcaccaatagccgcaggcaatccaaaacccatggctccaagaccccctgaggtcaaccactgcctcggtctcttgtacttgtaaaactgcgcagcccacatttgatgctgcccaaccccagtactaacaatagcatctccattagtcaactcatcaagaacctcgatagcatgctgcggagaaatcgcgtcctggaatgtcttgtaacccaatggaaacttgtgtttctgcacattaatctcttctctccaacctccaagatcaaacttaccctccactcctttctcctccaaaatcatattaattcccttcaaggccaacttcaaatccgcgcaaaccgacacgtgcgcctgcttgttcttcccaatctcggcagaatcaatatcaatgtgaacaatcttagccctactagcaaaagcctcaagcttcccagtaacacggtcatcaaaccttaccccaaaggcaagcaacaaatcactattgtcaacagcatagttagcataaacagtaccatgcatacccagcatctgaagggaatattcatcaccaataggaaaagttccaagacccattaaagtgctagcaacgggaataccagtgagttcaacaaagcgcctcaattcagcactggaattcaaactgccaccgccgacgtagagaacgggcttttgggcctccatgatgagtctgacaatgtgttccaattgggcctcggcggggggcctgggcagcctggcgaggtaaccggggaggttaacgggctcgtcccaattaggcacggcgagttgctgctgaacgtctttgggaatgtcgatgaggaccggaccggggcggccggaggtggcgacgaagaaagcctcggcgacgacgcgggggatgtcgtcgacgtcgaggatgaggtagttgtgcttcgtgatggatctgctcacctccacgatcggggtttcttggaaggcgtcggtgccgatcatccggcgggcgacctggccggtgatggcgacgactgggacgctgtccattaaagcgtcggcgaggccgctcacgaggttggtggcgccggggccggaggtggcaatgcagacgccggggaggccggaggaacgcgcgtagccttcggcggcgaagacgccgccctgctcgtggcgcgggagcacgttgcggatggcggcggagcgcgtgagcgcctggtggatctccatcgacgcaccgccggggtacgcgaacaccgtcgtcacgccctgcctctccagcgcctccacaaggatgtccgcgcccttgcgaggttcgccggaggcgaaccgtgacacgaagggctccgtggtcggcgcttccttggtgaagggcgccgccgtggggggtttggagatggaacatttgattttgagagcgtggttgggtttggtgagggtttgatgagagagagggagggtggatctagtaatgcgtttggggaaggtggggtgtgaagaggaagaagagaatcgggtggttctggaagcggtggccgccattgtgttgtgtggcatggttatacttcaaaaactgcacaacaagcctagagttagtacctaaacagtaaatttacaacagagagcaaagacacatgcaaaaatttcagccataaaaaaagttataatagaatttaaagcaaaagtttcattttttaaacatatatacaaacaaactggatttgaaggaagggattaattcccctgctcaaagtttgaattcctattgtgacctatactcgaataaaattgaagcctaaggaatgtatgagaaacaagaaaacaaaacaaaactacagacaaacaagtacaattacaaaattcgctaaaattctgtaatcaccaaaccccatctcagtcagcacaaggcccaaggtttattttgaaataaaaaaaaagtgattttatttctcataagctaaaagaaagaaaggcaattatgaaatgatttcgactagatctgaaagtccaacgcgtattccgcagatattaaagaaagagtagagtttcacatggatcctagatggacccagttgaggaaaaagcaaggcaaagcaaaccagaagtgcaagatccgaaattgaaccacggaatctaggatttggtagagggagaagaaaagtaccttgagaggtagaagagaagagaagagcagagagatatatgaacgagtgtgtcttggtctcaactctgaagcgatacgagtttagaggggagcattgagttccaatttatagggaaaccgggtggcaggggtgagttaatgacggaaaagcccctaagtaacgagattggattgtgggttagattcaaccgtttgcatccgcggcttagattggggaagtcagagtgaatctcaaccgttgactgagttgaaaattgaatgtagcaaccaattgagccaaccccagcctttgccctttgattttgatttgtttgttgcatactttttatttgtcttctggttctgactctctttctctcgtttcaatgccaggttgcctactcccacaccactcacaagaagattctactgttagtattaaatattttttaatgtattaaatgatgaatgcttttgtaaacagaacaagactatgtctaataagtgtcttgcaacattttttaagaaattaaaaaaaatatatttattatcaaaatcaaatgtatgaaaaatcatgaataatataattttatacatttttttaaaaaatcttttaatttcttaattaatatcttaaaaataatgattaatatttaacccaaaataattagtatgattggtaaggaagatatccatgttatgtttggatgtgagtttgatctagagcaaagcttactagagtcgaccgatccgtcgacggcgcg

SEQ ID NO: 4 - DNA, 40 bp, Artificial Sequence
  Oligonucleotide primer BM5
gccggggtaccggcgcgcccgatcatccggatatagttcc

SEQ ID NO: 5 - DNA, 40 bp, Artificial Sequence
  Oligonucleotide primer BM6
gccggggtaccggcgcgccgttctatagtgtcacctaatc

SEQ ID NO: 6 - DNA, 8911 bp, Artificial Sequence
  Plasmid pDN10
  misc_feature (3740)..(3740): n is a, c, g, or t
cggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccggtaccgggccccccctcgaggtcgacggtatcgataagcttgatatcgaattcctgcagcccgggggatccactagttctagagcggcccgcgccgtcgacggatataatgagccgtaaacaaagatgattaagtagtaattaatacgtactagtaaaagtggcaaaagataacgagaaagaaccaatttctttgcattcggccttagcggaaggcatatataagctttgattattttatttagtgtaatgatttcgtacaaccaaagcatttatttagtactctcacacttgtgtcgcggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaagtgtgttattaacccttctcttcgaggatccaagcttggcgcgggccgccaccgcggtggggtcgactctagtaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgttggactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataaccatgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatatcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccgggtac

SEQ ID NO: 7 - DNA, 2478 bp, Glycine max
ggtttgtttggtgtgagtgaatagggatcagggatgtggaggctgaagatagcagatggaggaaatgatccatacatattcagcacaaacaatttcgttgggaggcagacatgggagtttgatcctgaagcaggcagtccagaggaacgggcccaggttgaagcagctcgtcagcatttctaccacaaccgcttcaaggtcaagccctgcgctgacctcctttggcgttttcaggttctcagagaaaataacttcaaacaaacaattcctcgtgtgactatagaagatggagaggaaatcacataccaaaaagtcacaagcgccgtcagaaggggcgcacaccaccttgcggcactgcagacctctgatggccattggcctgctcaaattgcaggtcctctcttctttcttcctcccttggttttttgtatgtatattacaggaaatcttgaatcagtatttccagaagaacatcgcaaagaaattcttcgttacacatattatcaccagaatgaagacggaggatggggactacacatagagggtcatagcactatgttttgtactgcactgaactatatatgcatgcgaatgcttggagaaggacctaatggaggtcatgacaatgcttgtgctagagcaagaaagtggattcgagatcatggtggtgtaacacatataccttcatggggaaaaacttggctttcgatactcggtgtatttgattggtgcggaagcaacccaatgcccccagagttttggatccttccatcttttcttcctatgcatccagctaagatgtggtgttactgtcgattggtatacatgcctatgtcttacttatatgggaagaggtttgtgggtccaatcacaccactcatcttacaattaagagaagagttgtttactcaaccttatgaaaaagttaattggaagaaagcgcgtcaccaatgtgcaaaggaagatctttactatccccatcctttgatacaagacctaatatgggatagtttatacatattcactgaaccgctacttactcgttggcctttcaacaagttgattagagaaaaggcccttcaagtaactatgaaacatattcattatgaagatgagactagtcgatacataaccattggttgtgtggaaaaggttttatgtatgcttgcttgttgggtggaagatccaaacggagatgctttcaagaagcatcttgcaagggtcccagattacttatgggtttctgaagatggaatgaccatgcagagttttggtagccaagaatgggatgctggctttgctgttcaagctttgcttgccactaacataattgaagaaattggtcctacgtttgcaaaaggacatgatttcatcaagaagtctcaggtgaaggataatccttttggagattttaaaagtatgcatcgtcatatttctaaagggtcttggacattctctgatcaagaccatggatggcaagtttctgattgcactgcagaaggtttaaagtgttgtctacttctatcaatgttgccaccagagattgtgggagaaaagatggaacctgaaagattatacgattcagtcaatgtcttgttgtcgcttcagagtaaaaaaggtggtttagcagcatgggagcctgcaggagctcaagagtggttagaattactcaatcccacagaattttttgcggacattgtagttgaacatgaatatgttgagtgcactggatctgcaatccaagctttagttttgttcaagaagctatatccaggacataggaagaaagagatagaaaatttcattaccaatgcagttcgattccttgaagatacacaaacagctgatggttcatggtatggaaattggggagtttgcttcacttatggctcttggtttgcacttggaggtctagcagctgctggtaagacttacaccaattgtgctgccattcgcaaagccgttaaatttctacttacaacacaaagagaggacggtggatggggagagagttatctttcaagcccaaaaaagatatatgtacctctagaaggaagccgatcaaatgttgtacatacagcatgggctcttatgggactaattcatgctggacaggcggatagagaccccatgcctcttcaccgtgctgcaaagttgctcattaattctcagttggaagagggtgattggccccaacaggaaatcacgggagtattcatgaaaaattgcatgttgcattatccaatgtacagagatatttatccaatgtgggctctagctgaatatcgaaggcgggttccattgccttccactgaagtttaatttagaatggtttgagcacgaaaaggcaaaggcattttcattaagattgaggcaaataagttgtgtgtaatcaagcttaatcaattttttcatattcctatgtttatttcctacatatattggtagaaaaattatttcaaaaaaaaaaaaaaaaaa

SEQ ID NO: 8 - DNA, 30 bp, Artificial Sequence
  Oligonucleotide primer BM7
gcggccgcatgtggaggctgaagatagcag

SEQ ID NO: 9 - DNA, 31 bp, Artificial Sequence
  Oligonucleotide primer BM8
gcggccgcttaaacttcagtggaaggcaatg

SEQ ID NO: 10 - DNA, 2305 bp, Glycine max
  misc_feature (1)..(2305): Product of amplifying src3c.pk024.m11 with BM7 and BM8
gcggccgcatgtggaggctgaagatagcagatggaggaaatgatccatacatattcagcacaaacaatttcgttgggaggcagacatgggagtttgatcctgaagcaggcagtccagaggaacgggcccaggttgaagcagctcgtcagcatttctaccacaaccgcttcaaggtcaagccctgcgctgacctcctttggcgttttcaggttctcagagaaaataacttcaaacaaacaattcctcgtgtgactatagaagatggagaggaaatcacataccaaaaagtcacaagcgccgtcagaaggggcgcacaccaccttgcggcactgcagacctctgatggccattggcctgctcaaattgcaggtcctctcttctttcttcctcccttggttttttgtatgtatattacaggaaatcttgaatcagtatttccagaagaacatcgcaaagaaattcttcgttacacatattatcaccagaatgaagacggaggatggggactacacatagagggtcatagcactatgttttgtactgcactgaactatatatgcatgcgaatgcttggagaaggacctaatggaggtcatgacaatgcttgtgctagagcaagaaagtggattcgagatcatggtggtgtaacacatataccttcatggggaaaaacttggctttcgatactcggtgtatttgattggtgcggaagcaacccaatgcccccagagttttggatccttccatcttttcttcctatgcatccagctaagatgtggtgttactgtcgattggtatacatgcctatgtcttacttatatgggaagaggtttgtgggtccaatcacaccactcatcttacaattaagagaagagttgtttactcaaccttatgaaaaagttaattggaagaaagcgcgtcaccaatgtgcaaaggaagatctttactatccccatcctttgatacaagacctaatatgggatagtttatacatattcactgaaccgctacttactcgttggcctttcaacaagttgattagagaaaaggcccttcaagtaactatgaaacatattcattatgaagatgagactagtcgatacataaccattggttgtgtggaaaaggttttatgtatgcttgcttgttgggtggaagatccaaacggagatgctttcaagaagcatcttgcaagggtcccagattacttatgggtttctgaagatggaatgaccatgcagagttttggtagccaagaatgggatgctggctttgctgttcaagctttgcttgccactaacataattgaagaaattggtcctacgtttgcaaaaggacatgatttcatcaagaagtctcaggtgaaggataatccttttggagattttaaaagtatgcatcgtcatatttctaaagggtcttggacattctctgatcaagaccatggatggcaagtttctgattgcactgcagaaggtttaaagtgttgtctacttctatcaatgttgccaccagagattgtgggagaaaagatggaacctgaaagattatacgattcagtcaatgtcttgttgtcgcttcagagtaaaaaaggtggtttagcagcatgggagcctgcaggagctcaagagtggttagaattactcaatcccacagaattttttgcggacattgtagttgaacatgaatatgttgagtgcactggatctgcaatccaagctttagttttgttcaagaagctatatccaggacataggaagaaagagatagaaaatttcattaccaatgcagttcgattccttgaagatacacaaacagctgatggttcatggtatggaaattggggagtttgcttcacttatggctcttggtttgcacttggaggtctagcagctgctggtaagacttacaccaattgtgctgccattcgcaaagccgttaaatttctacttacaacacaaagagaggacggtggatggggagagagttatctttcaagcccaaaaaagatatatgtacctctagaaggaagccgatcaaatgttgtacatacagcatgggctcttatgggactaattcatgctggacaggcggatagagaccccatgcctcttcaccgtgctgcaaagttgctcattaattctcagttggaagagggtgattggccccaacaggaaatcacgggagtattcatgaaaaattgcatgttgcattatccaatgtacagagatatttatccaatgtgggctctagctgaatatcgaaggcgggttccattgccttccactgaagtttaagcggccgc

SEQ ID NO: 11 - protein, 762 aa, Glycine max
  MISC_FEATURE (1)..(762): translation of nt 9-2294 of SEQ ID NO10
MWRLKIADGGNDPYIFSTNNFVGRQTWEFDPEAGSPEERAQVEAARQHFYHNRFKVKPCADLLWRFQVLRENNFKQTIPRVTIEDGEEITYQKVTSAVRRGAHHLAALQTSDGHWPAQIAGPLFFLPPLVFCMYITGNLESVFPEEHRKEILRYTYYHQNEDGGWGLHIEGHSTMFCTALNYICMRMLGEGPNGGHDNACARARKWIRDHGGVTHIPSWGKTWLSILGVFDWCGSNPMPPEFWILPSFLPMHPAKMWCYCRLVYMPMSYLYGKRFVGPITPLILQLREELFTQPYEKVNWKKARHQCAKEDLYYPHPLIQDLIWDSLYIFTEPLLTRWPFNKLIREKALQVTMKHIHYEDETSRYITIGCVEKVLCMLACWVEDPNGDAFKKHLARVPDYLWVSEDGMTMQSFGSQEWDAGFAVQALLATNIIEEIGPTFAKGHDFIKKSQVKDNPFGDFKSMHRHISKGSWTFSDQDHGWQVSDCTAEGLKCCLLLSMLPPEIVGEKMEPERLYDSVNVLLSLQSKKGGLAAWEPAGAQEWLELLNPTEFFADIVVEHEYVECTGSAIQALVLFKKLYPGHRKKEIENFITNAVRFLEDTQTADGSWYGNWGVCFTYGSWFALGGLAAAGKTYTNCAAIRKAVKFLLTTQREDGGWGESYLSSPKKIYVPLEGSRSNVVHTAWALMGLIHAGQADRDPMPLHRAAKLLINSQLEEGDWPQQEITGVFMKNCMLHYPMYRDIYPMWALAEYRRRVPLPSTEV

SEQ ID NO: 12 - DNA, 11208 bp, Artificial Sequence
  Plasmid PHP20767
  misc_feature (6037)..(6037): n is a, c, g, or t
cggcgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccggtaccgggccccccctcgaggtcgacggtatcgataagcttgatatcgaattcctgcagcccgggggatccactagttctagagcggcccgcgccgtcgacggatataatgagccgtaaacaaagatgattaagtagtaattaatacgtactagtaaaagtggcaaaagataacgagaaagaaccaatttctttgcattcggccttagcggaaggcatatataagctttgattattttatttagtgtaatgatttcgtacaaccaaagcatttatttagtactctcacacttgtgtcgcggccgcttaaacttcagtggaaggcaatggaacccgccttcgatattcagctagagcccacattggataaatatctctgtacattggataatgcaacatgcaatttttcatgaatactcccgtgatttcctgttggggccaatcaccctcttccaactgagaattaatgagcaactttgcagcacggtgaagaggcatggggtctctatccgcctgtccagcatgaattagtcccataagagcccatgctgtatgtacaacatttgatcggcttccttctagaggtacatatatcttttttgggcttgaaagataactctctccccatccaccgtcctctctttgtgttgtaagtagaaatttaacggctttgcgaatggcagcacaattggtgtaagtcttaccagcagctgctagacctccaagtgcaaaccaagagccataagtgaagcaaactccccaatttccataccatgaaccatcagctgtttgtgtatcttcaaggaatcgaactgcattggtaatgaaattttctatctctttcttcctatgtcctggatatagcttcttgaacaaaactaaagcttggattgcagatccagtgcactcaacatattcatgttcaactacaatgtccgcaaaaaattctgtgggattgagtaattctaaccactcttgagctcctgcaggctcccatgctgctaaaccaccttttttactctgaagcgacaacaagacattgactgaatcgtataatctttcaggttccatcttttctcccacaatctctggtggcaacattgatagaagtagacaacactttaaaccttctgcagtgcaatcagaaacttgccatccatggtcttgatcagagaatgtccaagaccctttagaaatatgacgatgcatacttttaaaatctccaaaaggattatccttcacctgagacttcttgatgaaatcatgtccttttgcaaacgtaggaccaatttcttcaattatgttagtggcaagcaaagcttgaacagcaaagccagcatcccattcttggctaccaaaactctgcatggtcattccatcttcagaaacccataagtaatctgggacccttgcaagatgcttcttgaaagcatctccgtttggatcttccacccaacaagcaagcatacataaaaccttttccacacaaccaatggttatgtatcgactagtctcatcttcataatgaatatgtttcatagttacttgaagggccttttctctaatcaacttgttgaaaggccaacgagtaagtagcggttcagtgaatatgtataaactatcccatattaggtcttgtatcaaaggatggggatagtaaagatcttcctttgcacattggtgacgcgctttcttccaattaactttttcataaggttgagtaaacaactcttctcttaattgtaagatgagtggtgtgattggacccacaaacctcttcccatataagtaagacataggcatgtataccaatcgacagtaacaccacatcttagctggatgcataggaagaaaagatggaaggatccaaaactctgggggcattgggttgcttccgcaccaatcaaatacaccgagtatcgaaagccaagtttttccccatgaaggtatatgtgttacaccaccatgatctcgaatccactttcttgctctagcacaagcattgtcatgacctccattaggtccttctccaagcattcgcatgcatatatagttcagtgcagtacaaaacatagtgctatgaccctctatgtgtagtccccatcctccgtcttcattctggtgataatatgtgtaacgaagaatttctttgcgatgttcttctggaaatactgattcaagatttcctgtaatatacatacaaaaaaccaagggaggaagaaagaagagaggacctgcaatttgagcaggccaatggccatcagaggtctgcagtgccgcaaggtggtgtgcgccccttctgacggcgcttgtgactttttggtatgtgatttcctctccatcttctatagtcacacgaggaattgtttgtttgaagttattttctctgagaacctgaaaacgccaaaggaggtcagcgcagggcttgaccttgaagcggttgtggtagaaatgctgacgagctgcttcaacctgggcccgttcctctggactgcctgcttcaggatcaaactcccatgtctgcctcccaacgaaattgtttgtgctgaatatgtatggatcatttcctccatctgctatcttcagcctccacatgcggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaagtgtgttattaacccttctcttcgaggatccaagcttggcgcgggccgccaccgcggtggggtcgactctagtaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgttggactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataaccatgccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatatcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccgggtac